(19) United States
(12) Patent Application Publication
(10) Pub. No.: US 2006/0019885 A1
(43) Pub. Date: Jan. 26, 2006

(54) Modified bryodin 1 with reduced immunogenicity

(76) Inventors: Matthew Baker, Cambridge (GB); Francis J. Carr, Aberdeenshire (GB)

Correspondence Address: OLSON & HIERL, LTD., 20 NORTH WACKER DRIVE, 36TH FLOOR, CHICAGO, IL, 60606, US

(21) Appl. No.: 10/517,707

(22) PCT Filed: Jun. 10, 2003
(86) PCT No.: PCT/EP03/06055

§ 371 Date: Dec. 10, 2004

(30) Foreign Application Priority Data
Jun. 11, 2002 (EP) 02012911.0

(51) Int. Cl. A61K 38/17; C07K 14/47

(52) U.S. Cl. 514/12; 530/324

(57) ABSTRACT

The invention relates to the modification of bryodin 1 to result in bryodin 1 proteins that are substantially non-immunogenic or less immunogenic than any non-modified counterpart when used in vivo. The invention relates furthermore to T-cell epitope peptides derived from said nonmodified protein by means of which it is possible to create modified bryodin 1 variants with reduced immunogenicity.

### FIELD OF THE INVENTION

The present invention relates to polypeptides to be administered especially to humans and in particular for therapeutic use. The polypeptides are modified polypeptides whereby the modification results in a reduced propensity for the polypeptide to elicit an immune response upon administration to the human subject. The invention in particular relates to the modification of bryodin 1 to result in bryodin 1 proteins that are substantially non-immunogenic or less immunogenic than any non-modified counterpart when used in vivo. The invention relates furthermore to T-cell epitope peptides derived from said non-modified protein by means of which it is possible to create modified bryodin 1 variants with reduced immunogenicity.

### BACKGROUND OF THE INVENTION

There are many instances whereby the efficacy of a therapeutic protein is limited by an unwanted immune reaction to the therapeutic protein. Several mouse monoclonal antibodies have shown promise as therapies in a number of human disease settings but in certain cases have failed due to the induction of significant degrees of a human anti-murine antibody (HAMA) response [Schroff, R. W. et al (1985) Cancer Res. 45: 879-885; Shawler, D. L. et al (1985) J. Immunol. 135: 1530-1535]. For monoclonal antibodies, a number of techniques have been developed in attempt to reduce the HAMA response [WO 89/09622; EP 0239400; EP 0438310; WO 91/06667]. These recombinant DNA approaches have generally reduced the mouse genetic information in the final antibody construct whilst increasing the human genetic information in the final construct. Notwithstanding, the resultant “humanised” antibodies have, in several cases, still elicited an immune response in patients [Issacs J. D. (1990) Sem. Immunol. 2: 449,456; Rebello, P. R. et al (1999) Transplantation 68: 1417-1420].

Antibodies are not the only class of polypeptide molecule administered as a therapeutic agent against which an immune response may be mounted. Even proteins of human origin and with the same amino acid sequences as occur within humans can still induce an immune response in humans. Notable examples include the therapeutic use of granulocyte-macrophage colony stimulating factor [Wadhwa, M. et al (1999) Clin. Cancer Res. 5: 1353-1361] and interferon alpha 2 [Russo, D. et al (1996) Bri. J. Haem. 94: 300-305;. Stein, R. et al (1988) New Engl. J. Med. 318: 1409-1413] amongst others.

A principal factor in the induction of an immune response is the presence within the protein of peptides that can stimulate the activity of T-cells via presentation on MHC class II molecules, so-called “T-cell epitopes”. Such potential T-cell epitopes are commonly defined as any amino acid residue sequence with the ability to bind to MHC Class II molecules. Such T-cell epitopes can be measured to establish MHC binding. Implicitly, a “T-cell epitope” means an epitope which when bound to MHC molecules can be recognized by a T-cell receptor (TCR), and which can, at least in principle, cause the activation of these T-cells by engaging a TCR to promote a T-cell response. It is, however, usually understood that certain peptides which are found to bind to MHC Class II molecules may be retained in a protein sequence because such peptides are recognized as “self” within the organism into which the final protein is administered.

It is known, that certain of these T-cell epitope peptides can be released during the degradation of peptides, polypeptides or proteins within cells and subsequently be presented by molecules of the major histocompatability complex (MHC) in order to trigger the activation of T-cells. For peptides presented by MHC Class II, such activation of T-cells can then give rise, for example, to an antibody response by direct stimulation of B-cells to produce such antibodies.

MHC Class II molecules are a group of highly polymorphic proteins which play a central role in helper T-cell selection and activation. The human leukocyte antigen group DR (HLA-DR) are the predominant isotype of this group of proteins and are the major focus of the present invention. However, isotypes HLA-DQ and HLA-DP perform similar functions, hence the present invention is equally applicable to these. The MHC class II DR molecule is made of an alpha and a beta chain which insert at their C-termini through the cell membrane. Each hetero-dimer possesses a ligand binding domain which binds to peptides varying between 9 and 20 amino acids in length, although the binding groove can accommodate a maximum of 11 amino acids. The ligand binding domain is comprised of amino acids 1 to 85 of the alpha chain, and amino acids 1 to 94 of the beta chain. DQ molecules have recently been shown to have an homologous structure and the DP family proteins are also expected to be very similar. In humans approximately 70 different allotypes of the DR isotype are known, for DQ there are 30 different allotypes and for DP 47 different allotypes are known. Each individual bears two to four DR alleles, two DQ and two DP alleles. The structure of a number of DR molecules has been solved and such structures point to an open-ended peptide binding groove with a number of hydrophobic pockets which engage hydrophobic residues (pocket residues) of the peptide [Brown et al Nature (1993) 364: 33; Stern et al (1994) Nature 368: 215]. Polymorphism identifying the different allotypes of class II molecule contributes to a wide diversity of different binding surfaces for peptides within the peptide binding grove and at the population level ensures maximal flexibility with regard to the ability to recognize foreign proteins and mount an immune response to pathogenic organisms. There is a considerable amount of polymorphism within the ligand binding domain with distinct “families” within different geographical populations and ethnic groups. This polymorphism affects the binding characteristics of the peptide binding domain, thus different “families” of DR molecules will have specificities for peptides with different sequence properties, although there may be some overlap. This specificity determines recognition of Th-cell epitopes (Class II T-cell response) which are ultimately responsible for driving the antibody response to B-cell epitopes present on the same protein from which the Th-cell epitope is derived. Thus, the immune response to a protein in an individual is heavily influenced by T-cell epitope recognition which is a function of the peptide binding specificity of that individual's HLA-DR allotype. Therefore, in order to identify T-cell epitopes within a protein or peptide in the context of a global population, it is desirable to consider the binding properties of as diverse a set of HLA-DR allotypes as possible, thus covering as high a percentage of the world population as possible.

An immune response to a therapeutic protein proceeds via the MHC class II peptide presentation pathway. Here exogenous proteins are engulfed and processed for presentation in association with MHC class II molecules of the DR, DQ or DP type. MHC Class II molecules are expressed by professional antigen presenting cells (APCs), such as macrophages and dendritic cells amongst others. Engagement of a MHC class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response. The ability of a peptide to bind a given MHC class II molecule for presentation on the surface of an APC is dependent on a number of factors most notably its primary sequence. This will influence both its propensity for proteolytic cleavage and also its affinity for binding within the peptide binding cleft of the MHC class II molecule. The MHC class II/peptide complex on the APC surface presents a binding face to a particular T-cell receptor CTCR) able to recognize determinants provided both by exposed residues of the peptide and the MHC class II molecule.

In the art there are procedures for identifying synthetic peptides able to bind MHC class II molecules (e.g. WO98/52976 and WO/0034317). Such peptides may not function as T-cell epitopes in all situations, particularly, in vivo due to the processing pathways or other phenomena. T-cell epitope identification is the first step to epitope elimination. The identification and removal of potential T-cell epitopes from proteins has been previously disclosed. In the art methods have been provided to enable the detection of T-cell epitopes usually by computational means scanning for recognized sequence motifs in experimentally determined T-cell epitopes or alternatively using computational techniques to predict MHC class II-binding peptides and in particular DR-binding peptides.

WO98/52976 and WO/0034317 teach computational threading approaches to identifying polypeptide sequences with the potential to bind a sub-set of human MHC class II DR allotypes. In these teachings, predicted T-cell epitopes are removed by the use of judicious amino acid substitution within the primary sequence of the therapeutic antibody or non-antibody protein of both non-human and human derivation.

Other techniques exploiting soluble complexes of recombinant MHC molecules in combination with synthetic peptides and able to bind to T-cell clones from peripheral blood samples from human or experimental animal subjects have been used in the art Kern, F. et al (1998) Nature Medicine 4:975-978; Kwok, W. W. et al (2001) TRENDS in Immunol. 22:583-588]. These and other schemes including for example the use of whole proteins or synthetic peptides or variant molecules to the protein of interest may be screened for molecules with altered ability to bind or stimulate T-cells may equally be exploited in an epitope identification strategy.

As depicted above and as consequence thereof, it would be desirable to identify and to remove or at least to reduce T-cell epitopes from a given in principal therapeutically valuable but originally immunogenic peptide, polypeptide or protein.

One of these therapeutically valuable molecules is bryodin 1. The present invention provides for modified forms of bryodin 1 with one or more T cell epitopes removed. The sequence of bryodin 1 protein as given by Gawlak et al [Gawlak, S. et al (1997) Biochemistry 36:3095-3103] is depicted in single-letter code as follows:

DVSFRLSGATTTSYGVFIKNLREALPYERKVYNIPLLRSSISGSGRYTLL
HLTNYADETISVAVDVTNVYIMGLYAGDVSYFFNEASATEAAKFVFKDAK
KKVTLPYSGNYERLQTAAGKIRENIPLGLPALDSAITTLYYYTASSAASA
LLVLIQSTAESARYKFIEQQIGKRVDKTFLPSLATISLENNWSALSKQIQ
IASTNNGQFESPVVLIDGNNQRVSITNASARVVTSNIALLLNRNNIAAIG
EDISMTLIGFEHGLYGI

The bryodin 1 protein is single polypeptide of 267 amino acids with a molecular weight of approximately 29,000 Da. Bryodin 1 is a type 1 ribosome inactivating protein (RIP) originally isolated from the roots of the plant Bryonia dionica [U.S. Pat. No. 5,541,110]. There is considerable interest in this and other RIPs on account of their toxicity to living cells. In particular recombinant forms in fusion with cell-specific targeting domains (e.g. antibodies) have potential value in many therapeutic areas where the selective killing of particular cell populations is a desired outcome.

It is a particular objective of the present invention to provide modified bryodin 1 proteins in which the immune characteristic is modified by means of reduced numbers of potential T-cell epitopes.

Others have provided bryodin molecules and in particular recombinant bryodin 1 [U.S. Pat. No. 5,541,110; U.S. Pat. No. 5,932,447], but these teachings do not recognise the importance of T cell epitopes to the immunogenic properties of the protein nor have been conceived to directly influence said properties in a specific and controlled way according to the scheme of the present invention. By contrast, the PCT patent application WO00/34317 published Jun. 15, 2000 discloses a modified bryodin 1 molecule including substitutions at positions 5, 6, 18, 27, 111, 164, 216, 222, 237 and 249. The substitutions have been selected on the basis of an in silico motif matching tool and do not address the most biologically relevant MHC class II epitopes detected in a biological assay and which are for the first time disclosed herein. Moreover where the present invention discloses sequences to be considered as the biologically relevant epitopes in the subject molecule the inventors have recognized largely identical sequences in related proteins namely α-trichosanthin, α-momorcharin and β-momorcharin which accordingly by structural homology are relevant epitopes also in these proteins.

There is a continued need for bryodin 1 analogues with enhanced properties. Desired enhancements include alternative schemes and modalities for the expression and purification of the said therapeutic, but also and especially, improvements in the biological properties of the protein. There is a particular need for enhancement of the in vivo characteristics when administered to the human subject. In this regard, it is highly desired to provide bryodin 1 with reduced or absent potential to induce an immune response in the human subject.

### SUMMARY AND DESCRIPTION OF THE INVENTION

The present invention provides for modified forms of bryodin 1, in which the immune characteristic is modified by means of reduced numbers of potential T-cell epitopes.

The invention discloses sequences identified within the bryodin 1 primary sequence that are potential T-cell epitopes by virtue of MHC class II binding potential. This disclosure specifically pertains the bryodin 1 protein which inclusive of an N-terminal pro-peptide comprises 267 amino acid residues.

The present invention discloses the major regions of the bryodin 1 primary sequence that are immunogenic in man and thereby provide the critical information required to conduct modification of the sequence to eliminate or reduce the immunogenic effectiveness of these sites.

In one embodiment, synthetic peptides comprising the said immunogenic regions can be provided in a pharmaceutical composition for the purpose of promoting a tolerogenic response to the whole molecule.

In a further embodiment bryodin 1 molecules modified within the epitope regions herein disclosed can be used in pharmaceutical compositions.

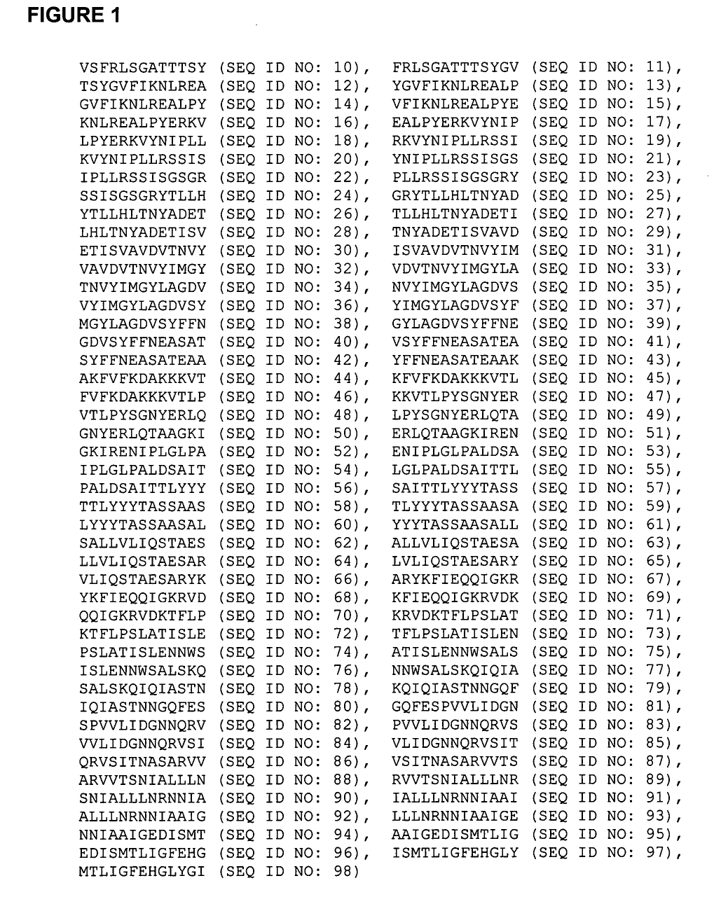

In summary the invention relates to the following issues:
using a panel of synthetic peptides in a naïve T-cell assay to map the immunogenic regions of bryodin 1;
bryodin 1 derived peptide sequences found to evoke a stimulation index of greater than around 2 in a naïve T-cell assay;
a molecule comprising a modified version of the bryodin 1 amino acid sequence and able to evoke a stimulation index of less than the value evoked by a wild-type bryodin 1 amino acid sequence in a T-cell proliferation assay using cells from a donor responsive to bryodin 1;
a modified molecule having the biological activity of bryodin 1 and being substantially non-immunogenic or less immunogenic than any non-modified molecule having the same biological activity when used in vivo;
an accordingly specified molecule, wherein said loss of immunogenicity is achieved by removing one or more T-cell epitopes derived from the originally non-modified molecule;
an accordingly specified molecule, wherein said loss of immunogenicity is achieved by reduction in numbers of MHC allotypes able to bind peptides derived from said molecule;
an accordingly specified molecule, wherein said originally present T-cell epitopes are MHC class II ligands or peptide sequences which show the ability to stimulate or bind T-cells via presentation on class II;
an accordingly specified molecule, wherein said peptide sequences are selected from the group as depicted in FIG. 1;
an accordingly specified molecule, wherein 1-9 amino acid residues, preferably one amino acid residue in any of the originally present T-cell epitopes are altered;
an accordingly specified molecule, wherein the alteration of the amino acid residues is substitution, addition or deletion of originally present amino acid(s) residue(s) by other amino acid residue(s) at specific position(s);
an accordingly specified molecule, wherein, if necessary, additionally further alteration usually by substitution, addition or deletion of specific amino acid(s) is conducted to restore biological activity of said molecule;
a peptide molecule of above sharing greater than 90% amino acid identity with any of the peptide sequences of FIG. 1;
a peptide molecule of above sharing greater than 80% amino acid identity with any of the peptide sequences of FIG. 1;
peptide sequences as above able to bind MHC class II;
an accordingly specified bryodin 1 molecule, wherein one or more of the amino acid substitutions is conducted at a position corresponding to any of the amino acids specified within FIG. 1;

an accordingly specified molecule wherein alteration is conducted at one or more residues from any or all of the string of contiguous residues of sequences (a), (b), (c), (d), or (e) as below wherein said sequences are derived from the bryodin 1 wild-type sequence where using single letter code;

(a) = RYTLLHLTNYADETISVAVDV (R1),
(b) = ATEAAKFVFKDAKKK (R2),
(c) = ERLQTAAKGKIRENIPLGLPALDSA (R3),
(d) = ITTLYYYTASSAASALLVLIQSTAESA (R4),
(e) = ATISLENNWSALSKQIQIAST (R5),

a peptide molecule comprising 13-15 consecutive residues from any of sequences (a), (b), (c), (d) or (e) above;
a peptide molecule comprising at least 9 consecutive residues from any of the sequences (a), (b), (c) (d) or (e) above;
a peptide molecule of above sharing greater than 90% amino acid identity with any of the peptide sequences derived from (a), (b), (d) or (e) above;
a peptide molecule of above sharing greater than 80% amino acid identity with any of the peptide sequences derived from (a), (b), (c) (d) or (e) above;
peptide sequences as above able to bind MHC class II;
an accordingly specified bryodin 1 molecule, wherein one or more of the amino acid substitutions is conducted at a position corresponding to any of the amino acids specified within any of sequences (a), (b), (c), (d) or (e) above;
an accordingly specified bryodin 1 molecule, wherein one or more of the amino acid substitutions is conducted at a position corresponding to any of the amino acids specified within sequence (a) and or (e) above;
an accordingly specified bryodin 1 molecule, wherein one or more of the amino acid substitutions is conducted at a position corresponding to any of the amino acids specified within sequence (a) and or (e) and additional substitutions made within sequence (c) and or (d) above;
a peptide sequence consisting of at least 9 consecutive amino acid residues of any of the sequences (a), (b), (c), (d) or (e) as specified above and its use for the manufacture of bryodin1, α-trichosanthin, α-momorcharin or β-momorcharin having substantially no or less immunogenicity than any non-modified molecule and having the biological activity of a type 1 RIP when used in vivo;
a pharmaceutical composition comprising any of the peptides or modified peptides of above having the activity of binding to MHC class II;
a DNA sequence or molecule which codes for any of said specified modified molecules as defined above and below;
a pharmaceutical composition comprising a modified molecule having the biological activity of bryodin 1;
a pharmaceutical composition as defined above and/or in the claims, optionally together with a pharmaceutically acceptable carrier, diluent or excipient;
a method for manufacturing a modified molecule having the biological activity of bryodin 1 as defined in any of the claims comprising the following steps: (i) determining the amino acid sequence of the polypeptide or part thereof; (ii) identifying one or more potential T-cell epitopes within the amino acid sequence of the protein by any method including determination of the binding of the peptides to MHC molecules using in vitro or in silico techniques or biological assays; (iii) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays; (iv) constructing such sequence variants by recombinant DNA techniques and testing said variants in order to identify one or more variants with desirable properties; and (v) optionally repeating steps (ii)-(iv);
an accordingly specified method, wherein step (iii) is carried out by substitution, addition or deletion of 1-9 amino acid residues in any of the originally present T-cell epitopes;
an accordingly specified method, wherein the alteration is made with reference to an homologous protein sequence and/or in silico modeling techniques;
an accordingly specified method, wherein step (ii) of above is carried out by the following steps: (a) selecting a region of the peptide having a known amino acid residue sequence; (b) sequentially sampling overlapping amino acid residue segments of predetermined uniform size and constituted by at least three amino acid residues from the selected region; (c) calculating MHC Class II molecule binding score for each said sampled segment by summing assigned values for each hydrophobic amino acid residue side chain present in said sampled amino acid residue segment; and (d) identifying at least one of said segments suitable for modification, based on the calculated MHC Class II molecule binding score for that segment, to change overall MHC Class II binding score for the peptide without substantially reducing therapeutic utility of the peptide; step (c) is preferably carried out by using a Böhm scoring function modified to include 12-6 van der Waal's ligand-protein energy repulsive term and ligand conformational energy term by (1) providing a first data base of MHC Class II molecule models; (2) providing a second data base of allowed peptide backbones for said MHC Class II molecule models; (3) selecting a model from said first data base; (4) selecting an allowed peptide backbone from said second data base; (5) identifying amino acid residue side chains present in each sampled segment; (6) determining the binding affinity value for all side chains present in each sampled segment; and repeating steps (1) through (5) for each said model and each said backbone;
a 13 mer T-cell epitope peptide having a potential MHC class II binding activity and created from non-modified bryodin 1, selected from the group as depicted in FIG. 1 and its use for the manufacture of bryodin 1 having substantially no or less immunogenicity than any non-modified molecule with the same biological activity when used in vivo;
a peptide sequence consisting of at least 9 consecutive amino acid residues of a 13 mer T-cell epitope peptide as derived from any of the sequences in FIG. 1 and its use for the manufacture of bryodin 1 having substantially no or less immunogenicity than any non-modified molecule and having the biological activity of a bryodin molecule when used in vivo.

a bryodin 1 molecule of structure according to Formula I:

X⁰DVSFRLSGATTTSYGVFIKNLREALPYERKVYNIPLLRSSISGSGRY
X¹X²LX³LTX⁴X⁵ADETX⁶SVAX⁷DX⁸TNVYIMGYLAGDVSYFFNEASAT
EAAKX⁹X¹⁰FKDAKKKX¹¹TLPYSGNYERKX¹²QTX¹³AX¹⁴X¹⁵X¹⁶
X¹⁷ENX¹⁸PLGX¹⁹PAX²⁰DSAX²¹TTX²²YX^{23TASSAASA}X²⁵X²⁶
X²⁷X²⁸IQSTAESARYKFIEQQIGKRVDKTFLPSLATX²⁹SX³⁰ENNWSA
X³¹SX³²QX³³QX³⁴ASTNNGQFESPVVLIDGNNQRVSITNASARVVTSN
IALLLNRNNIAAIGEDISMTLIGFEHGLYGI

wherein

X⁰ is hydrogen or a targeting moiety such as an antibody domain;

X¹ is most preferably A but G and P are also considered;

X² is most preferably M but A, G, P and I are also considered;

X³ is most preferably A but G and P are also considered;

X⁴ is most preferably P but Y is also considered;

X⁵ is most preferably T but S is also considered;

X⁶ is P;

X⁷ is most preferably A but P and G are also considered;

X⁸ is most preferably A but P and G are also considered;

X⁹ is most preferably A but P, G, H, D, E, N, Q, K, R, S and T are also considered;

X¹⁰ is most preferably A but P and G are also considered;

X¹¹ is most preferably A but P and G are also considered;

X¹² is most preferably A but P, S, T, H and K are also considered;

X¹³ is T;

X¹⁴ is H;

X¹⁵ is S;

X¹⁶ is most preferably A, but S, T, P, N, D, E, G, H, K and Q are also considered;

X¹⁷ is T;

X¹⁸ is most preferably A but P is also considered;

X¹⁹ is most preferably A but I, F, G, M, P, V, W and Y are also considered;

X²⁰ is most preferably F but P and W are also considered;

X²¹ is most preferably A but P and G are also considered;

X²² is most preferably G but A and P are also considered;

X²³ is most preferably G but A and P are also considered;

X²⁴ is most preferably A but P and G are also considered;

X²⁵ is most preferably A but P, G, S and T are also considered;

X²⁶ is most preferably A but I, M, S, T, P and G are also considered;

X²⁷ is most preferably A but G and P are also considered;

X²⁸ is most preferably S but A, G, P, T, H, D, N, Q, K and R are also condidered;

X²⁹ is most preferably T but A, G, S, P, H, K, R, D, E, N and Q are also considered;

X³⁰ is most preferably A but G, S, T, P, K, R, H, D, E, N and Q are also considered;

X³¹ is Q;

X³² is most preferably H but D, E, F, L, N, P, S, W and Y are also considered;

X³³ is most preferably T but A, G, P, D, E, H, K, R, N, Q, S and T are also considered;

X³⁴ is most preferably D,

and whereby simultaneously

X¹=T, X²=L, X³═H, X⁴═N, X⁵═Y, X⁶═I, X⁷═V, X⁸═V, X⁹═F, X¹⁰═V, X¹¹═V, X¹²=L, X¹³=A, X¹⁴=G, X¹⁵═K, X¹⁶═I, X¹⁷═R, X¹⁸═I, X¹⁹=L, X²⁰=L, X²¹═I, X²²═I, X²²=L, X²³═Y, X²⁴═Y, X²⁵=L, X²⁶=L, X²⁷═V, X²⁸=L, X²⁹═I, X³⁰=L, X³¹L, X³²═K, X³³═I and X³⁴═I are excluded.

The term “T-cell epitope” means according to the understanding of this invention an amino acid sequence which is able to bind MHC class II, able to stimulate T-cells and/or also to bind (without necessarily measurably activating) T-cells in complex with MHC class II.

The term “peptide” as used herein and in the appended claims, is a compound that includes two or more amino acids. The amino acids are linked together by a peptide bond (defined herein below). There are 20 different naturally occurring amino acids involved in the biological production of peptides, and any number of them may be linked in any order to form a peptide chain or ring. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Some peptides contain only a few amino acid units. Short peptides, e.g., having less than ten amino acid units, are sometimes referred to as “oligopeptides”. Other peptides contain a large number of amino acid residues, e.g. up to 100 or more, and are referred to as “polypeptides”. By convention, a “polypeptide” may be considered as any peptide chain containing three or more amino acids, whereas a “oligopeptide” is usually considered as a particular type of “short” polypeptide. Thus, as used herein, it is understood that any reference to a “polypeptide” also includes an oligopeptide. Further, any reference to a “peptide” includes polypeptides, oligopeptides, and proteins. Each different arrangement of amino acids forms different polypeptides or proteins. The number of polypeptides—and hence the number of different proteins-that can be formed is practically unlimited. “Alpha carbon (Cα)” is the carbon atom of the carbon-hydrogen (CH) component that is in the peptide chain. A “side chain” is a pendant group to Cα that can comprise a simple or complex group or moiety, having physical dimensions that can vary significantly compared to the dimensions of the peptide.

The invention may be applied to any bryodin 1 species of molecule with substantially the same primary amino acid sequences as those disclosed herein and would include therefore bryodin 1 molecules derived by genetic engineering means or other processes and may contain more or less than 267 amino acid residues.

The invention is conceived to overcome the practical reality that soluble proteins introduced with therapeutic intent in man trigger an immune response resulting in development of host antibodies that bind to the soluble protein. The present invention seeks to address this by providing bryodin 1 proteins with altered propensity to elicit an immune response on administration to the human host. According to the methods described herein, the inventors have discovered the regions of the bryodin 1 molecule comprising the critical T-cell epitopes driving the immune responses to this protein.

The general method of the present invention leading to the modified bryodin 1 comprises the following steps:

(a) determining the amino acid sequence of the polypeptide or part thereof;

(b) identifying one or more potential T-cell epitopes within the amino acid sequence of the protein by any method including determination of the binding of the peptides to MHC molecules using in vitro or in silico techniques or biological assays;
(c) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays. Such sequence variants are created in such a way to avoid creation of new potential T-cell epitopes by the sequence variations unless such new potential T-cell epitopes are, in turn, modified in such a way to substantially reduce or eliminate the activity of the T-cell epitope; and
(d) constructing such sequence variants by recombinant DNA techniques and testing said variants in order to identify one or more variants with desirable properties according to well known recombinant techniques.

The identification of potential T-cell epitopes according to step (b) can be carried out according to methods described previously in the art. Suitable methods are disclosed in WO 98/59244; WO 98/52976; WO 00/34317; WO 02/069232 and may be used to identify binding propensity of bryodin 1 derived peptides to an MHC class II molecule. In practice, the compositions embodied in the present invention have been derived with the concerted application of biological ex vivo human T-cell proliferation assays and a software tool exploiting the scheme outlined in WO 02/069232 and which is an embodiment of the present invention.

The software simulates the process of antigen presentation at the level of the peptide MHC class II binding interaction to provide a binding score for any given peptide sequence. Such a score is determined for many of the predominant MHC class II allotypes extant in the population. As this scheme is able to test any peptide sequence, the consequences of amino acid substitutions additions or deletions with respect to the ability of a peptide to interact with a MHC class II binding groove can be predicted. Consequently new sequence compositions can be designed which contain reduced numbers of peptides able to interact with the MHC class and thereby function as immmunogenic T-cell epitopes. Where the biological assay using any one given donor sample can assess binding to a maximum of 4 DR allotypes, the in silico process can test the same peptide sequence using >40 allotypes simultaneously. In practice this approach is able to direct the design of new sequence variants which are compromised in the their ability to interact with multiple MHC allotypes.

By way of an example of this in silico approach, the results of an analysis conducted on the entire bryodin 1 sequence is provided as FIG. 1. Therein are listed 13 mer peptide sequences derived from bryodin 1 detected to have the capability to bind one or more MHC class II allotypes with a significant binding score. Taken in its entirety, this dataset of 13 mer peptides is considered to provide with a high degree of certainty, the universe of permissible MHC class ligands for the bryodin 1 protein. For reasons such as the requirement for proteolytic processing of the complete bryodin 1 polypeptide and other physiologic steps leading to the presentation of bryodin 1 peptides in vivo, it would be clear that a relatively minor sub-set of the entire repertoire of peptides will have ultimate biological relevance. In order to further identify such biologically relevant peptides, the inventors have developed an approach exploiting ex vivo human T-cell proliferation assays.

This approach has proven to be a particularly effective method and is disclosed herein as an embodiment of the invention. The method can be applied to test part of the sequence, for example a sub-set of bryodin 1 peptides such as all or some of those listed in FIG. 1; or the method may be applied to test entire sequence. In the present studies, the method has involved the testing of overlapping bryodin 1 derived peptide sequences in a scheme so as to scan and test the entire bryodin 1 sequence (including peptides representing the N-terminal pro-pepeptide). The synthetic peptides are tested for their ability to evoke a proliferative response in human T-cell cultured in vitro. Where this type of approach is conducted using naïve human T-cells taken from healthy donors, the inventors have established that in the operation of such an assay, a stimulation index equal to or greater than 2.0 is a useful measure of induced proliferation. The stimulation index is conventionally derived by division of the proliferation score (e.g. counts per minute of radioactivity if using ³H-thymidine incorporation) measured to the test peptide by the score measured in cells not contacted with a test peptide.

The present studies have uncovered some 32 peptide sequences able to evoke a significant proliferative response (i.e. SI>2.0) in T-cells derived from at least one donor. Within this set of peptides, a further sub-set of peptides have been identified which evoke a significant proliferative response in 2 or more individual donor samples and for some of theses responses the magnitude of response has indeed been significantly higher than SI=2.0.

It is most preferred to provide a bryodin 1 molecule in which amino acid modification (e.g. a substitution) is conducted within the most immunogenic regions of the parent molecule. The inventors herein have discovered that the most immunogenic regions of the bryodin 1 molecule in man are confined to at least five regions R1- R5 encompassing residues 46-66; 88-102; 112-135; 136-162 and 178-204 comprising respectively amino acid sequences;

R1) RYTLLHLTNYADETISVAVDV;
R2) ATEAAKFVFKDAKKK;
R3) ERLQTAAGKIRENIPLGLPALDSA;
R4) ITTLYYYTASSAASALLVLIQSTAESA
and
R5) ATISLENNWSALSKQIQIAST.

These regions have been identified on the basis of giving SI>2 in one or more donor PBMC samples. For example epitope region R1 was proven to be reactive in 6 different donor samples representing over 28% of the donor samples screened. Similarly the R2 and R3 epitopes were reactive with 3 (14%) of donor samples tested, R4 with 5 (24%) of donor samples and R5 with 4 (19%) of donors tested. Taken together regions R1- R5 were reactive with 10 of the 21 (48%) donor PBMC samples tested covering a wide range of allotypic specificities.

The major preferred embodiments of the present invention comprise bryodin 1 molecules for which the MHC class II ligands identified within any of the epitopes R1- R5 are altered such as to eliminate binding or otherwise reduce the numbers of MHC allotypes to which the peptide can bind.

Where multiple potential epitopes are identified and in particular where a number of peptide sequences are found to be able to stimulate T-cells in a biological assay, cognisance may also be made of the structural features of the protein in relation to its propensity to evoke an immune response via the MHC class II presentation pathway. For example where the crystal structure of the protein of interest is known the crystallographic B-factor score may be analysed for evidence of structural disorder within the protein, a parameter suggested to correlate with the proximity to the biologically relevant immunodominant peptide epitopes [Dai G. et al (2001) J. Biological Chem. 276: 41913-41920). Such an analysis when conducted on the bryodin 1 crystal structure model [PDB ID: 1BRY, Gawlak, S. L., et al (1997) Biochemistry 36: 3095] suggests a high likelihood for multiple immunodominant epitopes with at least 4 discrete zones mapping to the medial position of areas with above average B-factor scores. Of the these 4 areas, 3 mapped to the N-terminal boundary of peptides shown to evoke a proliferative response in the naïve T-cell assay of EXAMPLE 2.

This data taken together with the data for the numbers of naïve donors responding to particular peptides enables a predicted ranking of the most immunodominant regions of the molecule. It is however recognised that in practice, each of these regions are considered immunogenic in man and therefore require modification under the scheme of the invention. Accordingly, with reference to the above defined sequence strings R1-R5, sequences may be ranked in the order {R1, R5}, {R3, R4}, R2; where {R1, R5} are considered the most immunogenic sequences and R2 relatively less immunogenic. Equal ranking is ascribed to those sequences in brackets. On this basis the most preferred bryodin 1 compositions under the scheme of the present involve modifications within epitope regions R1 and R5. Compositions containing in addition modifications within epitope regions R3 and R4 are also desired and optionally also additional substitutions within epitope region R2.

The disclosed peptide sequences herein represent the critical information required for the construction of modified bryodin 1 molecules in which one or more of these epitopes is compromised. Under the scheme of the present, the epitopes are compromised by mutation to result in sequences no longer able to function as T-cell epitopes. It is possible to use recombinant DNA methods to achieve directed mutagenesis of the target sequences and many such techniques are available and well known in the art. In practice a number of variant bryodin proteins will be produced and tested for the desired immune and functional characteristic.

Where it is the objective of this invention to modify the amino acid sequences of at least one or more of the above listed peptides from FIG. 1, it is most preferred to modify the sequence of one or more of the epitope regions R1- R5 identified above. There are herein disclosed suitable modifications which achieve the objective of reducing or eliminating the capabilities of the subject peptide sequence to function as a T-cell epitope and which may result in a bryodin 1 molecule with a reduced immunogenic potential when administered as a therapeutic to the human host.

For the elimination of T-cell epitopes, amino acid substitutions are preferably made at appropriate points within the peptide sequence predicted to achieve substantial reduction or elimination of the activity of the T-cell epitope. In practice an appropriate point will preferably equate to an amino acid residue binding within one of the pockets provided within the MHC class II binding groove. It is most preferred to alter binding within the first pocket of the cleft at the so-called P1 or P1 anchor position of the peptide. The quality of binding interaction between the P1 anchor residue of the peptide and the first pocket of the MHC class II binding groove is recognised as being a major determinant of overall binding affinity for the whole peptide. An appropriate substitution at this position of the peptide will be for a residue less readily accommodated within the pocket, for example, substitution to a more hydrophilic residue. Combinations of substitution within a single epitope may be contemplated and for example can be particularly appropriate where individually defined epitopes are in overlap with each other. Moreover, amino acid substitutions either singly within a given epitope or in combination within a single epitope may be made at positions not equating to the “pocket residues” with respect to the MHC class II binding groove, but at any point within the peptide sequence. Substitutions may be made with reference to an homologous structure or structural method produced using in silico techniques known in the art and may be based on known structural features of the molecule according to this invention. All such substitutions fall within the scope of the present invention.

Amino acid substitutions other than within the peptides identified above may be contemplated particularly when made in combination with substitution(s) made within a listed peptide. For example a change may be contemplated to restore structure or biological activity of the variant molecule. Such compensatory changes and changes to include deletion or addition of particular amino acid residues from the bryodin 1 polypeptide resulting in a variant with desired activity and in combination with changes in any of the disclosed peptides fall under the scope of the present.

One example of such a set of preferred modifications is provided by the disruption of the R1 epitope region. Complete elimination of all possible MHC ligands within this region is achieved by the a substitution set comprising the changes; T₄₉A, L₅₀M, H₅₂A, N₅₅P, Y₅₆T, I₆₁P, V₆₅A and V₆₇A. Such preferred changes either in isolation or in combination are an embodiment of the invention

Similarly, a preferred set of modifications achieving the disruption of the R2 epitope is provided by the substitution set F₉₉A, V₁₀₀A and V₁₀₈A. Such preferred changes either in isolation or in combination are an embodiment of the invention.

For epitope region R3 alternative substitution sets are defined based on knowledge of the key structural features of the molecule. It would be highly desired to construct a modified bryodin 1 molecule containing substitution at leucine residue 115 (L₁₁₅), as this residue can function as a P1 anchor for one MHC class II ligand identified within the R3 epitope. A preferred set of substitutions would accordingly comprise L₁₁₅A, I₁₂₂A, I₁₂₆A L₁₃₀A, L₁₃₃F and I₁₃₇A. However as L₁₁₅ is located to the floor of the binding cleft for RIP activity this substitution may compromise the functional activity of the molecule. An alternative set of substitutions can be defined which serve to disrupt the significant MHC ligands within the R3 epitope and yet which maintain L₁₁₅. Accordingly these substitutions comprise A₁₁₈T, G₁₂₀H, K₁₂₁S and R₁₂₃T and would be made in alternative to the dual changes L₁₁₅A and I₁₂₂A. All changes are an embodiment of the invention.

For epitope region R4, a preferred substitution set comprises the changes L₁₄₀G, Y₁₄₂G, Y₁₄₃A in combination with L₁₅₂A, L₁₅₃A, V₁₅₄A and L₁₅₅S. All changes either in isolation or in combination are embodiment of the invention.

A yet further example of a set of preferred modifications is provided by the disruption of the R5 epitope region using the changes comprising I₁₈₇T, L₁₈₉A, L₁₉₆Q, K₁₉₇H, I₂₀₀T and I₂₀₂D. All changes either in isolation or in combination are embodiment of the invention.

For nearly all of the above preferred substitutions alternative amino acids may be considered at any given position. The choices of alternative residue are however not un-limited and are confined to residues satisfying the broad objectives of reducing or eliminating the potential MHC peptide interaction and also being accommodated within the structure of the molecule; i.e. significant side chain clashes are avoided for most rotamers and or electrostatic or other contacts are either preserved or made. Examples of alternative residue choices which may be considered are provided in the bryodin 1 structure as depicted in the FORMULA 1.

From the foregoing it can be seen that according to this invention a number of variant bryodin 1 proteins can be produced and tested for the desired immune and functional characteristic and all such functional proteins are embodiments of the present invention. Moreover the modifications conducted have been demonstrated to result in peptide sequences not able to bind MHC class II molecules with the same affinity as the parental or “wild-type” (wt) peptide sequence using the predictive in silico MHC class II binding tool of WO02/069232.

The preferred molecules of this invention can be prepared in any of several ways but is most preferably conducted exploiting routine recombinant methods. It is a relatively facile procedure to use the protein sequences and information provided herein to deduce a polynucleotide (DNA) encoding any of the preferred protein sequences. This can be achieved for example using computer software tools such as the DNSstar software suite [DNAstar Inc, Madison, Wis., USA] or similar. Any such DNA sequence with the capability of encoding the preferred polypeptides of the present or significant homologues thereof, should be considered as embodiments of this invention.

As a general scheme, genes encoding any of the preferred bryodin 1 protein sequences can be made using gene synthesis and cloned into a suitable expression vector. In turn the expression vector is introduced into. a host cell and cells selected and cultured. The preferred molecules are purified from the culture medium and formulated into a preparation for therapeutic administration. Alternatively, a wild-type bryodin 1 gene sequence can be obtained for example following a cDNA cloning strategy using RNA prepared from the root tissues of the Bryonia plant. The wild-type gene can be used as a template for mutagenesis and construction preferred variant sequences. In this regard it is particularly convenient to use the strategy of “overlap extension PCR” as described by Higuchi et al [Higuchi et al (1988) Nucleic Acids Res. 16: 7351] although other methodologies and systems could be readily applied.

Constitution of the preferred bryodin 1 molecule may be achieved by recombinant DNA techniques and this includes bryodin 1 molecules fused with desired anti-body variable region domains or other targeting moieties. Methods for purifying and manipulating recombinant proteins including fusion proteins are well known in the art. Necessary techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991).

As will be clear to the person skilled in the art, multiple alternative sets of substitutions could be arrived at which achieve the objective of removing un-desired epitopes. The resulting sequences would however be recognised to be closely homologous with the specific compositions disclosed herein and therefore fall under the scope of the present invention.

In as far as this invention relates to modified bryodin 1, compositions containing such modified bryodin proteins or fragments of modified bryodipproteins and related compositions should be considered within the scope of the invention. In another aspect, the present invention relates to nucleic acids encoding modified bryodin entities. In a further aspect the present invention relates to methods for therapeutic treatment of humans using the modified bryodin 1 proteins. In this aspect the modified bryodin 1 protein may be linked with an antibody molecule or fragment of an antibody molecule. The linkage may be by means of a chemical cross-linker or the bryodin 1-antibody may be produced as a recombinant fusion protein. The fusion molecule may contain the modified bryodin 1 domain with antibody domain orientated towards the N-terminus of the fusion molecule although the opposite orientation may be contemplated.

Desired antibody specificities for linkage to the modified bryodin 1 molecule of the present include those directed towards internalising antigen determinants. Examples of this class of antigen are rare but would include the A33.antigen [Heath, J. K. et al (1997) Proc. Natl, Acad. Sci U.S.A. 94: 469-474] and the GA733-1 antigen [U.S. Pat. No. 5,840,854]. The carcinoembryonic antigen may also be contemplated for use and may be targeted by any of numerous antibodies but may include MFE23 [Chester, K. A. et al (1994) Lancet 343: 455], A5B7 [WO92/010159], T84.66 [U.S. Pat. No. 5,081,235] MN-14 [Hansen, H. J. et al (1993) Cancer 71: 3478-3485], COL-1 [U.S. Pat. No. 5,472,693] and others. Other desired specificities include antibodies directed to non-internalising antigens and this may include antigens such as the 40 kDa glycoprotein antigen as recognised by antibody KS¼ [Spearman et al (1987) J. Pharmacol. Exp. Therapeutics 241: 695-703] and other antibodies. Other antigens such as the epidermal growth factor receptor (HER1) or related receptors such as HE2 may be selected including anti-GD2 antibodies such as antibody 14.18 [U.S. Pat. No. 4,675,287; EP 0 192 657], or antibodies to the prostate specific membrane antigen [U.S. Pat. No. 6,107,090], the IL-2 receptor [U.S. Pat. No. 6,013,256], the Lewis Y determinant, mucin glycoproteins or others may be contemplated.

In all instances where a modified bryodin 1 protein is made in fusion with an antibody sequence it is most desired to use antibody sequences in which T cell epitopes or sequences able to bind MHC class II molecules or stimulate T cells or bind to T cells in association with MHC class II molecules have been removed.

A further embodiment of the present invention, the modified bryodin 1 protein may be linked to a non-antibody protein yet a protein able to direct a specific binding interaction to a particular target cell. Such protein moieties include a variety of polypeptide ligands for which there are specific cell surface receptors and include therefore numerous cytokines, peptide and polypeptide hormones and other biological response modifiers. Prominent examples include such proteins as vascular epithelial growth factor, epidermal growth factor, heregulin, the interleukins, interferons, tumour necrosis factor and other protein and glycoprotein molecules. Fusion proteins of these and other molecules with bryodin 1 of the present invention may be contemplated and may comprise the modified bryodin 1 moiety in either the N-terminal or C-terminal orientation with respect to the protein ligand domain. Equally, chemical cross-linking of the purified ligand to the modified bryodin 1 protein may be contemplated and within the scope of the present invention.

In a further embodiment the modified bryodin 1 protein of the present may be used as a complex containing a water soluble polymer such as hydroxypropylmethacrylamide or other polymers where the modified bryodin 1 protein is in covalent attachment to the polymer or in a non-covalent binding interaction with the polymer. Such an embodiment may additionally include an antigen binding domain such as an antibody or a fragment of an antibody in combination with the polymer bryodin 1 complex.

In a further aspect still, the invention relates to methods for therapeutic treatment using pharmaceutical preparations comprising peptide or derivative molecules with sequence identity or part identity with the sequences herein disclosed.

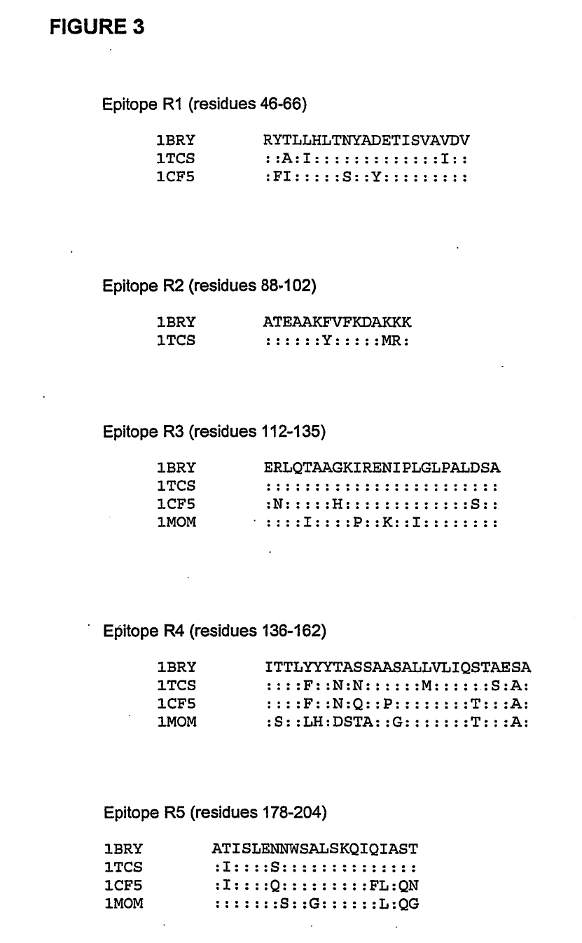

In a yet further aspect, the major immunogenic epitopes herein disclosed and relating to the bryodin 1 molecule are also shown herein to be present within the primary sequence of a number of other type 1 RIP proteins of which bryodin 1 is an example. Thus the proteins α-trichosanthin (1TCS), α-momorcharin (1MOM) and β-momorcharin (1CF5) and others may be shown by protein sequence analysis to contain sequence elements with identity or near identity to the immunogenic regions of the bryodin 1 molecule. FIG. 3 depicts sequence comparisons between bryodin 1 major epitopes and sequence elements from 1TCS, 1MOM and 1CF5 proteins. The present invention in so far as it relates to peptides and modified sequences derived from the bryodin 1 protein, where the identical or substantially similar sequences are identified within other proteins, these are considered equally to fall under the scope of the present. This is particularly true for some of the preferred mutation sets identified herein. For example the changes within R2 and R3 implemented in the bryodin 1 sequence may be applied for the removal of MHC class II ligands from the equivalent regions within the 1TCS sequence. Equally, the R1 changes in bryodin 1 comprising one or more of the substitutions T₄₉A, L₅₀M, H₅₂A, N₅₅P, Y₅₆T, I₆₁P, V₆₅A and V₆₇A can be applied to the equivalent regions with the proteins 1TCS and 1CFS. In the foregoing, numbering is according to the bryodin 1 sequence. A proportion of the preferred R4 and R5 changes may also be implemented within the RIP proteins 1TCS, 1CF5 and 1MOM and equally fall under the scope of the present invention.

In as far as this invention relates to modified bryodin 1, compositions containing such modified bryodin 1 proteins or fragments of modified bryodin 1 proteins and related compositions should be considered within the scope of the invention. A pertinent example in this respect could be development of peptide mediated tolerance induction strategies wherein one or more of the disclosed peptides is administered to a patient with immunotherapeutic intent. Accordingly, synthetic peptides molecules, for example one of more of those listed in FIG. 1 or more preferably sequences comprising all or part of any of the epitope regions R1- R5 as defined above. Such peptide are considered embodiments of the invention.

In another aspect, the present invention relates to nucleic acids encoding modified bryodin 1 entities.

The invention will now be illustrated by the experimental examples below. The invention is additionally illustrated by the figures described below:

FIG. 1 provides a list of peptide sequences in bryodin 1 with potential human MHC class II binding activty Peptides are 13-mers, amino acids are identified using single letter code

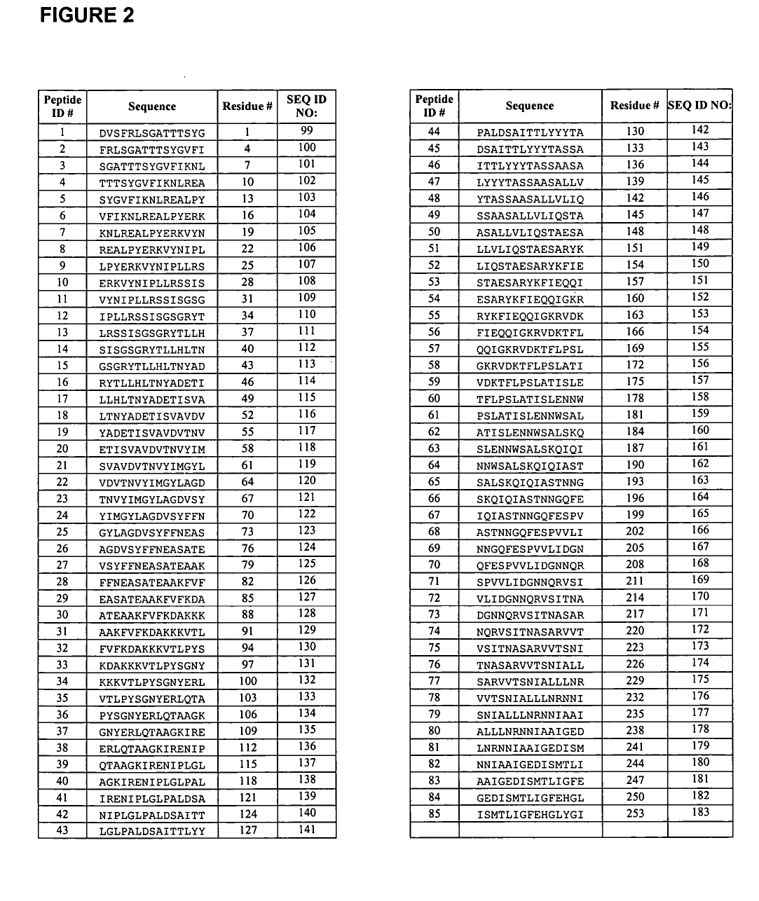

FIG. 2 provides a table of the bryodin 1 15-mer peptide sequences analysed using the naïve human in vitro T-cell assay of EXAMPLE 2. The peptide ID# and position of the N-terminal peptide residue within the bryodin 1 sequence is indicated

FIG. 3 indicates the sequence elements R1, R2, R3, R4 and R5 from the bryodin 1 (1BRY) sequence which give a stimulation index of 2.0 or greater in PBMC preparations from 2 or more donors PBMC using the naïve human in vitro T-cell assay of EXAMPLE 2. Corresponding sequences from related proteins α-trichosanthin (1TCS), α-momorcharin (1MOM) and β-momorcharin (1CF5) are shown beneath each bryodin 1 sequence. Sequences are identical to bryodin 1 except where indicated. Amino acids are depicted using single letter code.

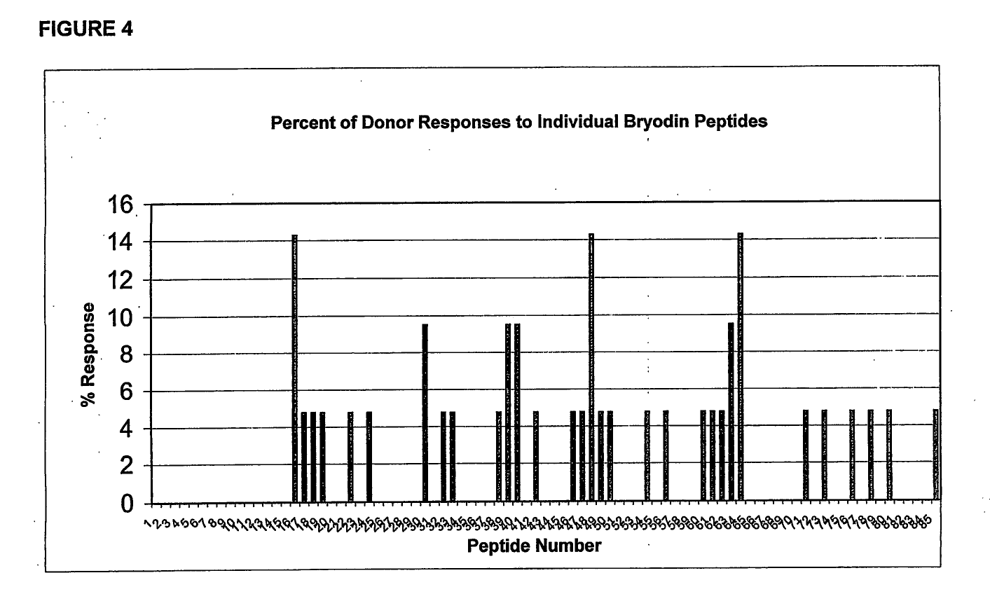

FIG. 4 shows the percent of donor responses to individual bryodin 1 peptides. The total number of 85 peptides were tested using PBMC preparations from 21 donor samples. A positive response is taken as an SI>2, epitope regions are identified where positive responses are seen in 2 or more donors.

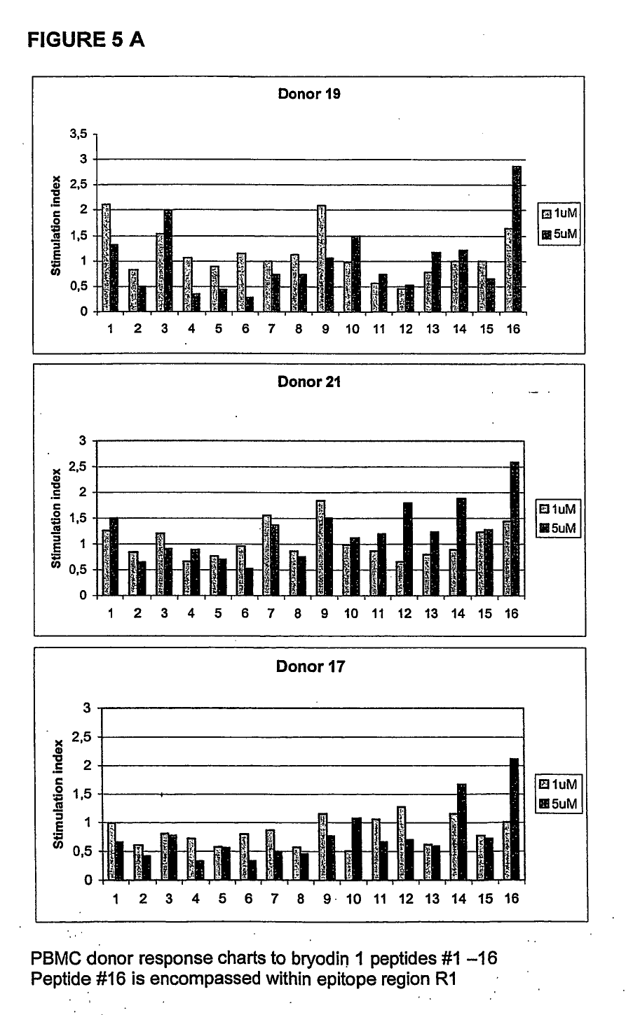

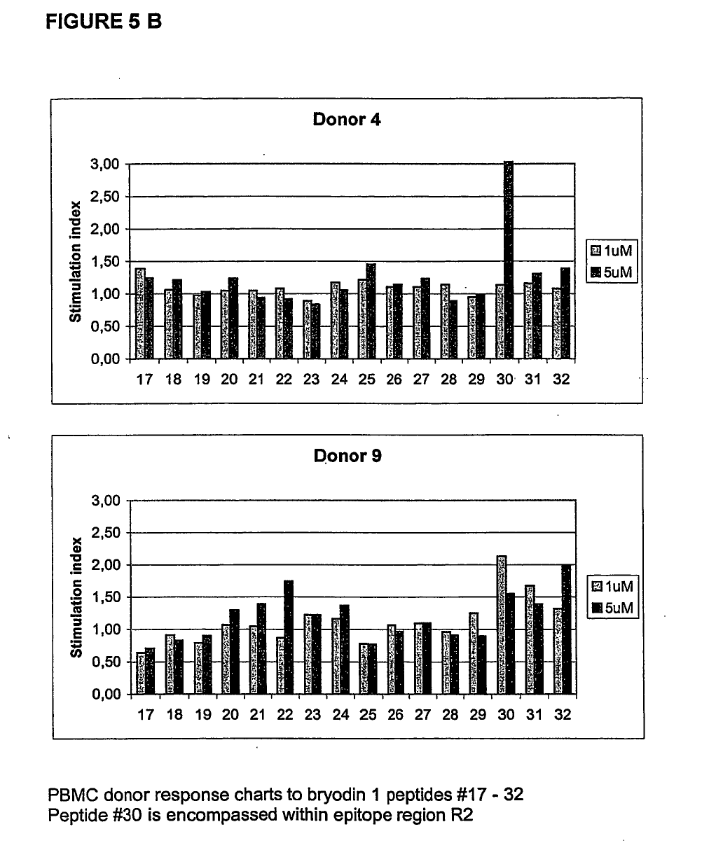

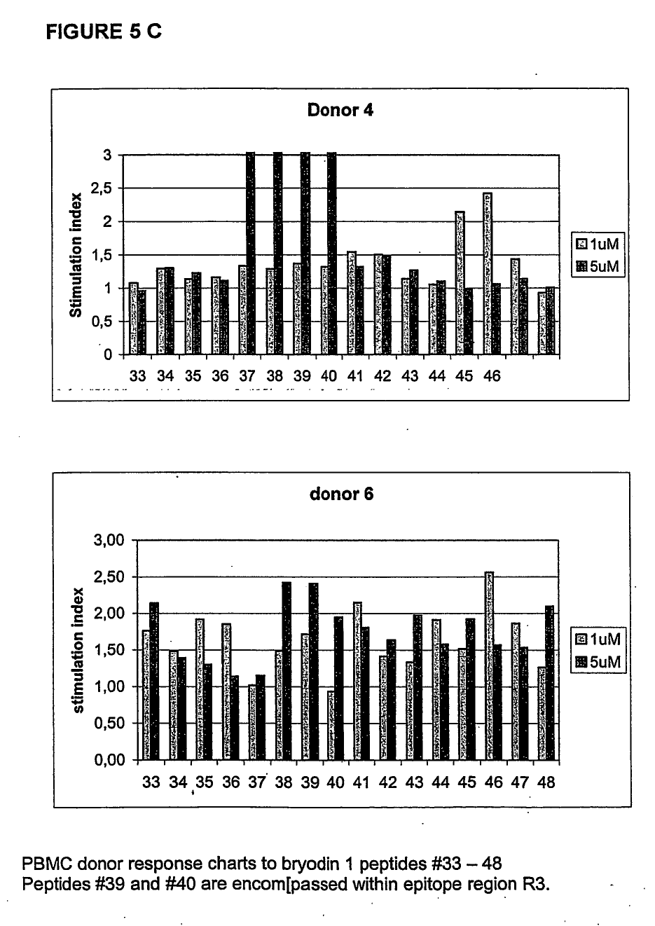

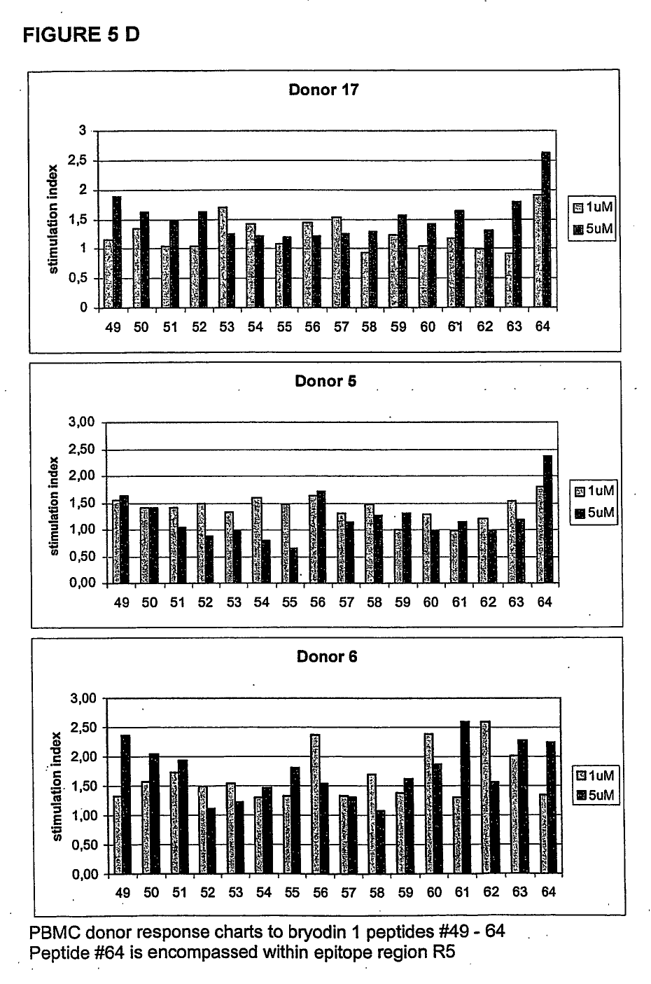

FIG. 5 shows representative stimulation Index (SI) plots from naïve human T-cell proliferation assays. Responses are shown for 1 μM and 5 μM concentrations of peptide. Each peak is the mean of a triplicate assay.

Panel A shows PBMC responses from 3 donor samples to bryodin 1 peptides encompassed within epitope region R1.

Panel B shows PBMC responses from 2 donor samples to bryodin 1 peptides encompassed within epitope region R2.

Panel C shows PBMC responses from 2 donor samples to bryodin 1 peptides encompassed within epitope region R3.

Panel D shows PBMC responses from 3 donor samples to bryodin 1 peptides encompassed within epitope region R5.

FIG. 6 is a depiction of the MHC class II ligands identified within epitope region R1. Ligands are identified using the in silico system of EXAMPLE 1. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

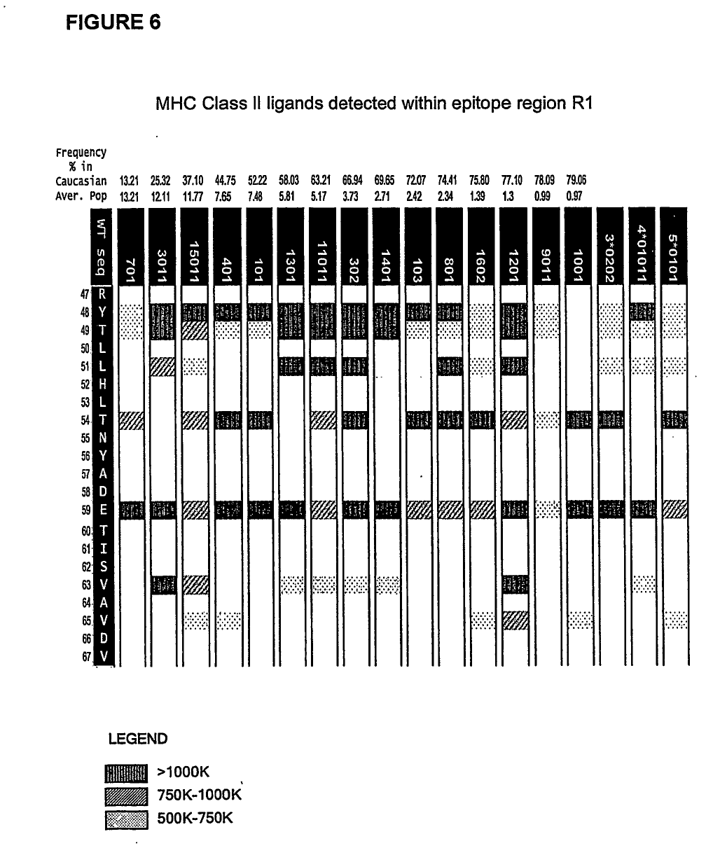

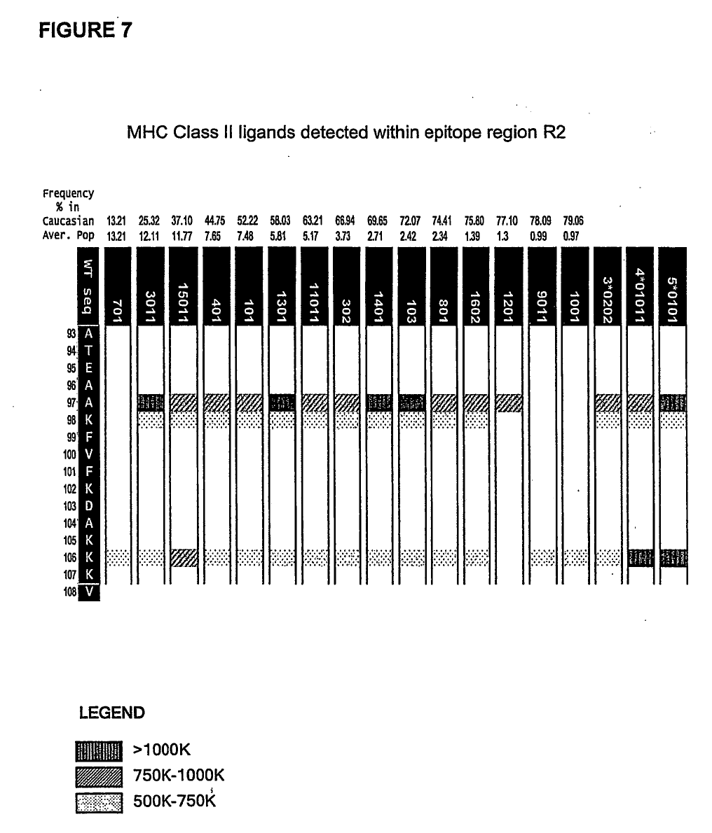

FIG. 7 is a depiction of the MHC class II ligands identified within epitope region R2. Ligands are identified using the in silico system of EXAMPLE 1. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

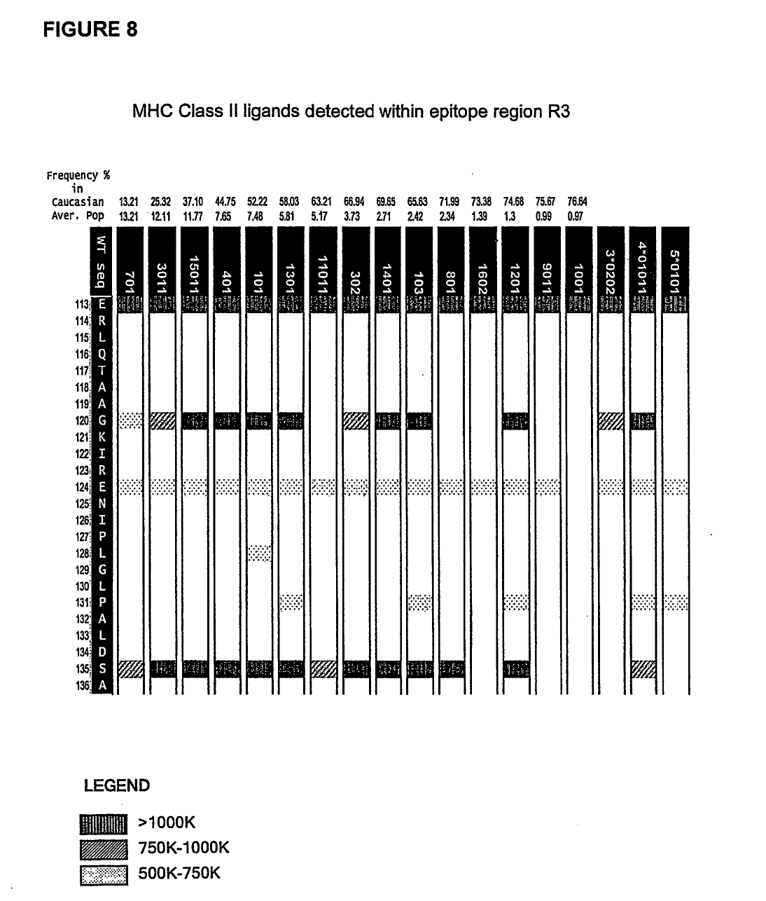

FIG. 8 is a depiction of the MHC class II ligands identified within epitope region R3. Ligands are identified using the in silico system of EXAMPLE 1. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

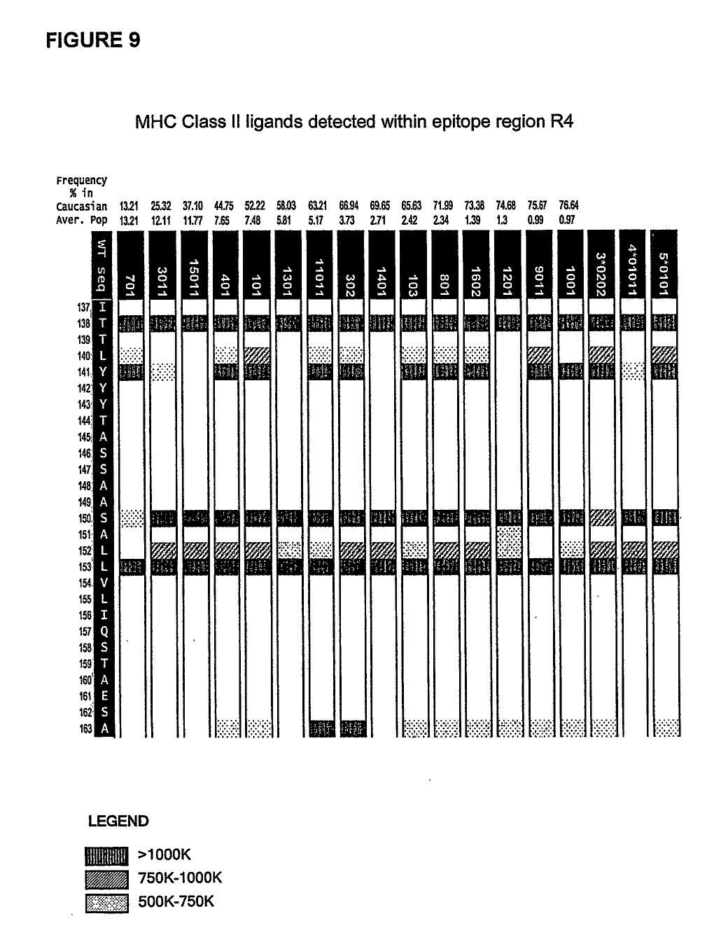

FIG. 9 is a depiction of the MHC class II ligands identified within epitope region R4. Ligands are identified using the in silico system of EXAMPLE 1. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

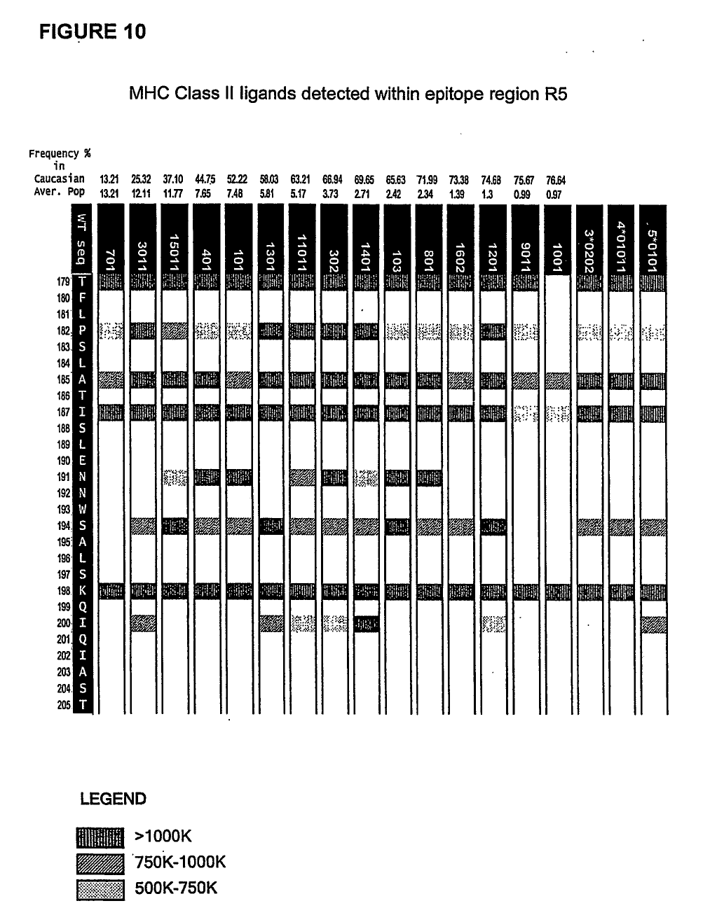

FIG. 10 is a depiction of the MHC class II ligands identified within epitope region R5. Ligands are identified using the in silico system of EXAMPLE 1. In this case the binding profile of 18 human DR allotypes are displayed as columns. The ligands detected are 13-mers and residue number 1 of each 13-mer is identified by a coloured block. The intensity of the binding interaction (High, Medium or Low) for each peptide with respect to each of the 18 allotypes is indicated according to the key displayed.

FORMULA 1 depicts a most preferred bryodin 1 structure featuring alternative substitutions which could be considered for incorporation into a bryodin 1 molecule with a reduced immunogenic potential.

### EXAMPLE 1

Method of Identifying Epitopes in Bryodin 1 Using an In Silico System for Conducting Peptide MHC Binding Analyses:

There are a number of factors that play important roles in determining the total structure of a protein or polypeptide. First, the peptide bond, i.e., that bond which joins the amino acids in the chain together, is a covalent bond. This bond is planar in structure, essentially a substituted amide. An “amide” is any of a group of organic compounds containing the grouping —CONH—.

The planar peptide bond linking Cα of adjacent amino acids may be represented as 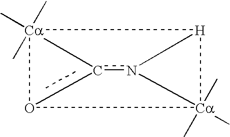
depicted below:

Because the O═C and the C—N atoms lie in a relatively rigid plane, free rotation does not occur about these axes. Hence, a plane schematically depicted by the interrupted line is sometimes referred to as an “amide” or “peptide plane” plane wherein lie the oxygen (O), carbon (C), nitrogen (N), and hydrogen (H) atoms of the peptide backbone. At opposite corners of this amide plane are located the Cα atoms. Since there is substantially no rotation about the O═C and C—N atoms in the peptide or amide plane, a polypeptide chain thus comprises a series of planar peptide linkages joining the Cα atoms. A second factor that plays an important role in defining the total structure or conformation of a polypeptide or protein is the angle of rotation of each amide plane about the common Cα linkage. The terms “angle of rotation” and “torsion angle” are hereinafter regarded as equivalent terms. Assuming that the O, C, N, and H atoms remain in the amide plane (which is usually a valid assumption, although there may be some slight deviations from planarity of these atoms for some conformations), these angles of rotation define the N and R polypeptide's backbone conformation, i.e., the structure as it exists between adjacent residues. These two angles are known as φ and ψ. A set of the angles φ₁, ψ₁, where the subscript i represents a particular residue of a polypeptide chain, thus effectively defines the polypeptide secondary structure. The conventions used in defining the φ, ψ angles, i.e., the reference points at which the amide planes form a zero degree angle, and the definition of which angle is φ, and which angle is ψ, for a given polypeptide, are defined in the literature. See, e.g., Ramachandran et al. Adv. Prot. Chem. 23:283-437 (1968), at pages 285-94, which pages are incorporated herein by reference.

The present method can be applied to any protein, and is based in part upon the discovery that in humans the primary Pocket 1 anchor position of MHC Class II molecule binding grooves has a well designed specificity for particular amino acid side chains. The specificity of this pocket is determined by the identity of the amino acid at position 86 of the beta chain of the MHC Class II molecule. This site is located at the bottom of Pocket 1 and determines the size of the side chain that can be accommodated by this pocket. Marshall, K. W., J. Immunol., 152:4946-4956 (1994). If this residue is a glycine, then all hydrophobic aliphatic and aromatic amino acids (hydrophobic aliphatics being: valine, leucine, isoleucine, methionine and aromatics being: phenylalanine, tyrosine and tryptophan) can be accommodated in the pocket, a preference being for the aromatic side chains. If this pocket residue is a valine, then the side chain of this amino acid protrudes into the pocket and restricts the size of peptide side chains that can be accommodated such that only hydrophobic aliphatic side chains can be accommodated. Therefore, in an amino acid residue sequence, wherever an amino acid with a hydrophobic aliphatic or aromatic side chain is found, there is the potential for a MHC Class II restricted T-cell epitope to be present. If the side-chain is hydrophobic aliphatic, however, it is approximately twice as likely to be associated with a T-cell epitope than an aromatic side chain (assuming an approximately even distribution of Pocket 1 types throughout the global population).

A computational method embodying the present invention profiles the likelihood of peptide regions to contain T-cell epitopes as follows:

(1) The primary sequence of a peptide segment of predetermined length is scanned, and all hydrophobic aliphatic and aromatic side chains present are identified. (2) The hydrophobic aliphatic side chains are assigned a value greater than that for the aromatic side chains; preferably about twice the value assigned to the aromatic side chains, e.g., a value of 2 for a hydrophobic aliphatic side chain and a value of 1 for an aromatic side chain. (3) The values determined to be present are summed for each overlapping amino acid residue segment (window) of predetermined uniform length within the peptide, and the total value for a particular segment (window) is assigned to a single amino acid residue at an intermediate position of the segment (window), preferably to a residue at about the midpoint of the sampled segment (window). This procedure is repeated for each sampled overlapping amino acid residue segment (window). Thus, each amino acid residue of the peptide is assigned a value that relates to the likelihood of a T-cell epitope being present in that particular segment (window). (4) The values calculated and assigned as described in Step 3, above, can be plotted against the amino acid coordinates of the entire amino acid residue sequence being assessed. (5) All portions of the sequence which have a score of a predetermined value, e.g., a value of 1, are deemed likely to contain a T-cell epitope and can be modified, if desired.

This particular aspect of the present invention provides a general method by which the regions of peptides likely to contain T-cell epitopes can be described. Modifications to the peptide in these regions have the potential to modify the MHC Class II binding characteristics.

According to another aspect of the present invention, T-cell epitopes can be predicted with greater accuracy by the use of a more sophisticated computational method which takes into account the interactions of peptides with models of MHC Class II alleles. The computational prediction of T-cell epitopes present within a peptide according to this particular aspect contemplates the construction of models of at least 42 MHC Class II alleles based upon the structures of all known MHC Class II molecules and a method for the use of these models in the computational identification of T-cell epitopes, the construction of libraries of peptide backbones for each model in order to allow for the known variability in relative peptide backbone alpha carbon (Cα) positions, the construction of libraries of amino-acid side chain conformations for each backbone dock with each model for each of the 20 amino-acid alternatives at positions critical for the interaction between peptide and MHC Class II molecule, and the use of these libraries of backbones and side-chain conformations in conjunction with a scoring function to select the optimum backbone and side-chain conformation for a particular peptide docked with a particular MHC Class II molecule and the derivation of a binding score from this interaction.

Models of MHC Class II molecules can be derived via homology modeling from a number of similar structures found in the Brookhaven Protein Data Bank (“PDB”). These may be made by the use of semi-automatic homology modeling software (Modeller, Sali A. & Blundell T L., 1993. J. Mol Biol 234:779-815) which incorporates a simulated annealing function, in conjunction with the CHARMm force-field for energy minimisation (available from Molecular Simulations Inc., San Diego, Calif.). Alternative modeling methods can be utilized as well.

The present method differs significantly from other computational methods which use libraries of experimentally derived binding data of each amino-acid alternative at each position in the binding groove for a small set of MHC Class II molecules (Marshall, K. W., et al., Biomed. Pept. Proteins Nucleic Acids, 1(3):157-162) (1995) or yet other computational methods which use similar experimental binding data in order to define the binding characteristics of particular types of binding pockets within the groove, again using a relatively small subset of MHC Class II molecules, and then ‘mixing and matching’ pocket types from this pocket library to artificially create further ‘virtual’ MHC Class II molecules (Stumiolo T., et al., Nat. Biotech, 17(6): 555-561 (1999). Both prior methods suffer the major disadvantage that, due to the complexity of the assays and the need to synthesize large numbers of peptide variants, only a small number of MHC Class II molecules can be experimentally scanned. Therefore the first prior method can only make predictions for a small number of MHC Class II molecules. The second prior method also makes the assumption that a pocket lined with similar amino-acids in one molecule will have the same binding characteristics when in the context of a different Class II allele and suffers further disadvantages in that only those MHC Class II molecules can be ‘virtually’ created which contain pockets contained within the pocket library. Using the modeling approach described herein, the structure of any number and type of MHC Class II molecules can be deduced, therefore alleles can be specifically selected to be representative of the global population. In addition, the number of MHC Class II molecules scanned can be increased by making further models further than having to generate additional data via complex experimentation.

The use of a backbone library allows for variation in the positions of the Cα atoms of the various peptides being scanned when docked with particular MHC Class II molecules. This is again in contrast to the alternative prior computational methods described above which rely on the use of simplified peptide backbones for scanning amino-acid binding in particular pockets. These simplified backbones are not likely to be representative of backbone conformations found in ‘real’ peptides leading to inaccuracies in prediction of peptide binding. The present backbone library is created by superposing the backbones of all peptides bound to MHC Class II molecules found within the Protein Data Bank and noting the root mean square (RMS) deviation between the Cα atoms of each of the eleven anmino-acids located within the binding groove. While this library can be derived from a small number of suitable available mouse and human structures (currently 13), in order to allow for the possibility of even greater variability, the RMS figure for each C-α position is increased by 50%. The average Cα position of each amino-acid is then determined and a sphere drawn around this point whose radius equals the RMS deviation at that position plus 50%. This sphere represents all allowed Cα positions.

Working from the Cα with the least RMS deviation (that of the amino-acid in Pocket 1 as mentioned above, equivalent to Position 2 of the 11 residues in the binding groove), the sphere is three-dimensionally gridded, and each vertex within the grid is then used as a possible location for a Cα of that amino-acid. The subsequent amide plane, corresponding to the peptide bond to the subsequent amino-acid is grafted onto each of these Cαs and the φ and ψ angles are rotated step-wise at set intervals in order to position the subsequent Cα. If the subsequent Cα falls within the ‘sphere of allowed positions’ for this Cα than the orientation of the dipeptide is accepted, whereas if it falls outside the sphere then the dipeptide is rejected.

This process is then repeated for each of the subsequent Cα positions, such that the peptide grows from the Pocket 1 Cα ‘seed’, until all nine subsequent Cαs have been positioned from all possible permutations of the preceding Cαs. The process is then repeated once more for the single Cα preceding pocket 1 to create a library of backbone Cα positions located within the binding groove.

The number of backbones generated is dependent upon several factors: The size of the ‘spheres of allowed positions’; the fineness of the gridding of the ‘primary sphere’ at the Pocket 1 position; the fineness of the step-wise rotation of the φ and ψ angles used to position subsequent Cαs. Using this process, a large library of backbones can be created. The larger the backbone library, the more likely it will be that the optimum fit will be found for a particular peptide within the binding groove of an MHC Class II molecule. Inasmuch as all backbones will not be suitable for docking with all the models of MHC Class II molecules due to clashes with amino-acids of the binding domains, for each allele a subset of the library is created comprising backbones which can be accommodated by that allele.

The use of the backbone library, in conjunction with the models of MHC Class II molecules creates an exhaustive database consisting of allowed side chain conformations for each amino-acid in each position of the binding groove for each MHC Class II molecule docked with each allowed backbone. This data set is generated using a simple steric overlap function where a MHC Class II molecule is docked with a backbone and an amino-acid side chain is grafted onto the backbone at the desired position. Each of the rotatable bonds of the side chain is rotated step-wise at set intervals and the resultant positions of the atoms dependent upon that bond noted. The interaction of the atom with atoms of side-chains of the binding groove is noted and positions are either accepted or rejected according to the following criteria: The sum total of the overlap of all atoms so far positioned must not exceed a predetermined value. Thus the stringency of the conformational search is a function of the interval used in the step-wise rotation of the bond and the pre-determined limit for the total overlap. This latter value can be small if it is known that a particular pocket is rigid, however the stringency can be relaxed if the positions of pocket side-chains are known to be relatively flexible. Thus allowances can be made to imitate variations in flexibility within pockets of the binding groove. This conformational search is then repeated for every amino-acid at every position of each backbone when docked with each of the MHC Class II molecules to create the exhaustive database of side-chain conformations.

A suitable mathematical expression is used to estimate the energy of binding between models of MHC Class II molecules in conjunction with peptide ligand conformations which have to be empirically derived by scanning the large database of backbone/side-chain conformations described above. Thus a protein is scanned for potential T-cell epitopes by subjecting each possible peptide of length varying between 9 and 20 amino-acids (although the length is kept constant for each scan) to the following computations: An MHC Class II molecule is selected together with a peptide backbone allowed for that molecule and the side-chains corresponding to the desired peptide sequence are grafted on. Atom identity and interatomic distance data relating to a particular side-chain at a particular position on the backbone are collected for each allowed conformation of that amino-acid (obtained from the database described above). This is repeated for each side-chain along the backbone and peptide scores derived using a scoring function. The best score for that backbone is retained and the process repeated for each allowed backbone for the selected model. The scores from all allowed backbones are compared and the highest score is deemed to be the peptide score for the desired peptide in that MHC Class II model. This process is then repeated for each model with every possible peptide derived from the protein being scanned, and the scores for peptides versus models are displayed.

In the context of the present invention, each ligand presented for the binding affinity calculation is an amino-acid segment selected from a peptide or protein as discussed above. Thus, the ligand is a selected stretch of amino acids about 9 to 20 amino acids in length derived from a peptide, polypeptide or protein of known sequence. The terms “amino acids” and “residues” are hereinafter regarded as equivalent terms. The ligand, in the form of the consecutive amino acids of the peptide to be examined grafted onto a backbone from the backbone library, is positioned in the binding cleft of an MHC Class II molecule from the MHC Class II molecule model library via the coordinates of the C″-α atoms of the peptide backbone and an allowed conformation for each side-chain is selected from the database of allowed conformations. The relevant atom identities and interatomic distances are also retrieved from this database and used to calculate the peptide binding score. Ligands with a high binding affinity for the MHC Class II binding pocket are flagged as candidates for site-directed mutagenesis. Amino-acid substitutions are made in the flagged ligand (and hence in the protein of interest) which is then retested using the scoring function in order to determine changes which reduce the binding affinity below a predetermined threshold value. These changes can then be incorporated into the protein of interest to remove T-cell epitopes.

Binding between the peptide ligand and the binding groove of MHC Class II molecules involves non-covalent interactions including, but not limited to: hydrogen bonds, electrostatic interactions, hydrophobic (lipophilic) interactions and Van der Walls interactions. These are included in the peptide scoring function as described in detail below.

It should be understood that a hydrogen bond is a non-covalent bond which can be formed between polar or charged groups and consists of a hydrogen atom shared by two other atoms. The hydrogen of the hydrogen donor has a positive charge where the hydrogen acceptor has a partial negative charge. For the purposes of peptide/protein interactions, hydrogen bond donors may be either nitrogens with hydrogen attached or hydrogens attached to oxygen or nitrogen. Hydrogen bond acceptor atoms may be oxygens not attached to hydrogen, nitrogens with no hydrogens attached and one or two connections, or sulphurs with only one connection. Certain atoms, such as oxygens attached to hydrogens or imine nitrogens (e.g. C═NH) may be both hydrogen acceptors or donors. Hydrogen bond energies range from 3 to 7 Kcal/mol and are much stronger than Van der Waal's bonds, but weaker than covalent bonds. Hydrogen bonds are also highly directional and are at their strongest when the donor atom, hydrogen atom and acceptor atom are co-linear.

Electrostatic bonds are formed between oppositely charged ion pairs and the strength of the interaction is inversely proportional to the square of the distance between the atoms according to Coulomb's law. The optimal distance between ion pairs is about 2.8 Å. In protein/peptide interactions, electrostatic bonds may be formed between arginine, histidine or lysine and aspartate or glutamate. The strength of the bond will depend upon the pKa of the ionizing group and the dielectric constant of the medium although they are approximately similar in strength to hydrogen bonds.

Lipophilic interactions are favorable hydrophobic-hydrophobic contacts that occur between he protein and peptide ligand. Usually, these will occur between hydrophobic amino acid side chains of the peptide buried within the pockets of the binding groove such that they are not exposed to solvent. Exposure of the hydrophobic residues to solvent is highly unfavorable since the surrounding solvent molecules are forced to hydrogen bond with each other forming cage-like clathrate structures. The resultant decrease in entropy is highly unfavorable. Lipophilic atoms may be sulphurs which are neither polar nor hydrogen acceptors and carbon atoms which are not polar.

Van der Waal's bonds are non-specific forces found between atoms which are 3-4 Å apart. They are weaker and less specific than hydrogen and electrostatic bonds. The distribution of electronic charge around an atom changes with time and, at any instant, the charge distribution is not symmetric. This transient asymmetry in electronic charge induces a similar asymmetry in neighboring atoms. The resultant attractive forces between atoms reaches a maximum at the Van der Waal's contact distance but diminishes very rapidly at about 1 Å to about 2 Å. Conversely, as atoms become separated by less than the contact distance, increasingly strong repulsive forces become dominant as the outer electron clouds of the atoms overlap. Although the attractive forces are relatively weak compared to electrostatic and hydrogen bonds (about 0.6 Kcal/mol), the repulsive forces in particular may be very important in determining whether a peptide ligand may bind successfully to a protein.

In one embodiment, the Böhm scoring function (SCORE1 approach) is used to estimate the binding constant. (Böhm, H. J., J. Comput Aided Mol. Des., 8(3):243-256 (1994) which is hereby incorporated in its entirety). In another embodiment, the scoring function (SCORE2 approach) is used to estimate the binding affinities as an indicator of a ligand containing a T-cell epitope (Böhm, H. J., J. Comput Aided Mol. Des., 12(4):309-323 (1998) which is hereby incorporated in its entirety). However, the Böhm scoring functions as described in the above references are used to estimate the binding affinity of a ligand to a protein where it is already known that the ligand successfully binds to the protein and the protein/ligand complex has had its structure solved, the solved structure being present in the Protein Data Bank (“PDB”). Therefore, the scoring function has been developed with the benefit of known positive binding data. In order to allow for discrimination between positive and negative binders, a repulsion term must be added to the equation. In addition, a more satisfactory estimate of binding energy is achieved by computing the lipophilic interactions in a pairwise manner rather than using the area based energy term of the above Böhm functions.

Therefore, in a preferred embodiment, the binding energy is estimated using a modified Böhm scoring function. In the modified Böhm scoring function, the binding energy between protein and ligand (ΔG_{bind}) is estimated considering the following parameters: The reduction of binding energy due to the overall loss of translational and rotational entropy of the ligand (ΔG₀); contributions from ideal hydrogen bonds (ΔG_{hb}) where at least one partner is neutral; contributions from unperturbed ionic interactions (ΔG_{ionic}); lipophilic interactions between lipophilic ligand atoms and lipophilic acceptor atoms (ΔGₗᵢₚₒ); the loss of binding energy due to the freezing of internal degrees of freedom in the ligand, i.e., the freedom of rotation about each C-C bond is reduced (ΔGᵣₒₜ); the energy of the interaction between the protein and ligand (E_{VdW}). Consideration of these terms gives equation 1:
(ΔG_{bind})=(ΔG₀)+(αG_{hb}×N_{hb})+(αG_{ionic}×N_{ionic})+(ΔGₗᵢₚₒ×Nₗᵢₚₒ)+(ΔGᵣₒₜ+Nᵣₒₜ)+(E_{VdW})

Where N is the number of qualifying interactions for a specific term and, in one embodiment, ΔG₀, ΔG_{hb}, ΔG_{ionic}, ΔGₗᵢₚₒ, and ΔGᵣₒₜ are constants which are given the values: 5.4, −4.7, −4.7, −0.17, and 1.4, respectively.

The term N_{hb} is calculated according to equation 2:
N_{hb}=Σ_{h-bonds}f(ΔR, Δα)×f(N_{neighb})×f_{pcs}

f(ΔR, Δα) is a penalty function which accounts for large deviations of hydrogen bonds from ideality and is calculated according to equation 3:
f(ΔR,Δ−□)=f1(ΔR)×f2(Δα)

$$Where \text{:}$$

$$f1 ( Δ R ) = 1 if Δ R <= TOL$$

$$or = 1 - ( Δ R - TOL ) / 0.4 if Δ R <= 0.4 + TOL \\ or = 0 if Δ R > 0.4 + TOL \\ And \text{:} \\ f2 ( Δα ) = 1 if Δα < 30 ° or = 1 - ( Δ α - 30 ) / 50 if Δα <= 80 ° \\ or = 0 if Δα > 80 °$$

TOL is the tolerated deviation in hydrogen bond length=0.25 Å ΔR is the deviation of the H—O/N hydrogen bond length from the ideal value=1.9 Å Δα is the deviation of the hydrogen bond angle ∠_{N/O—H..O/N} from its idealized value of 180°

f(N_{neighb}) distinguishes between concave and convex parts of a protein surface and therefore assigns greater weight to polar interactions found in pockets rather than those found at the protein surface. This function is calculated according to equation 4 below:
f(N_{neighb})=(N_{neighb}/N_{neighb,0})^{α} where α=0.5

N_{neighb} is the number of non-hydrogen protein atoms that are closer than 5 Å to any given protein atom.
N_{neighb,0} is a constant=25

f_{pcs} is a function which allows for the polar contact surface area per hydrogen bond and therefore distinguishes between strong and weak hydrogen bonds and its value is determined according to the following criteria:
f_{pcs}=β when Aₚₒₗₐᵣ/N_{NB}<10 Å²
or f_{pcs}=1 when Aₚₒₗₐᵣ/N_{HB}10 Å²

Aₚₒₗₐᵣ is the size of the polar protein-ligand contact surface

N_{HB} is the number of hydrogen bonds

β is a constant whose value=1.2

For the implementation of the modified Böhm scoring function, the contributions from ionic interactions, ΔG_{ionic}, are computed in a similar fashion to those from hydrogen bonds described above since the same geometry dependency is assumed.

The term Nₗᵢₚₒ is calculated according to equation 5 below:
Nₗᵢₚₒ=Σ_{IL}f(r_{IL})

f(r_{IL}) is calculated for all lipophilic ligand atoms, 1, and all lipophilic protein atoms, L, according to the following criteria:
f(r_{IL})=1 when r_{IL}<=R1f(r_{IL})=(r_{IL}−R1)/(R2−R1) when R2<r_{IL}>R1
f(r_{IL})=0 when r_{IL}>=R2
Where: R1=r₁^{vdw}+r_{L}^{vdw}+0.5
and R2=R1+3.0

and r₁^{vdw} is the Van der Waal's radius of atom 1

and r_{L}^{vdw} is the Van der Waal's radius of atom L

The term Nᵣₒₜ is the number of rotable bonds of the amino acid side chain and is taken to be the number of acyclic sp³-sp³ and sp³-sp² bonds. Rotations of terminal −CH₃ or —NH₃ are not taken into account.

The final term, E_{VdW}, is calculated according to equation 6 below:
E_{VdW}=ε₁ε₂((r₁^{vdw}+rᵣ^{vdw})¹²/r¹²−(r₁^{vdw}+r₂^{vdw}⁶/r⁶), where:

ε₁ and ε₂ are constants dependant upon atom identity

r₁^{vdw}+r₂^{vdw} are the Van der Waal's atomic radii

r is the distance between a pair of atoms.

With regard to Equation 6, in one embodiment, the constants ε₁ and ε₂ are given the atom values: C: 0.245, N: 0.283, O: 0.316, S: 0.316, respectively (i.e. for atoms of Carbon, Nitrogen, Oxygen and Sulphur, respectively). With regards to equations 5 and 6, the Van der Waal's radii are given the atom values C: 1.85, N: 1.75, O: 1.60, S: 2.00 Å.

It should be understood that all predetermined values and constants given in the equations above are determined within the constraints of current understandings of protein ligand interactions with particular regard to the type of computation being undertaken herein. Therefore, it is possible that, as this scoring function is refined further, these values and constants may change hence any suitable numerical value which gives the desired results in terms of estimating the binding energy of a protein to a ligand may be used and hence fall within the scope of the present invention.

As described above, the scoring function is applied to data extracted from the database of side-chain conformations, atom identities, and interatomic distances. For the purposes of the present description, the number of MHC Class II molecules included in this database is 42 models plus four solved structures. It should be apparent from the above descriptions that the modular nature of the construction of the computational method of the present invention means that new models can simply be added and scanned with the peptide backbone library and side-chain conformational search function to create additional data sets which can be processed by the peptide scoring function as described above. This allows for the repertoire of scanned MHC Class II molecules to easily be increased, or structures and associated data to be replaced if data are available to create more accurate models of the existing alleles.

The present prediction method can be calibrated against a data set comprising a large number of peptides whose affinity for various MHC Class II molecules has previously been experimentally determined. By comparison of calculated versus experimental data, a cut of value can be determined above which it is known that all experimentally determined T-cell epitopes are correctly predicted.

It should be understood that, although the above scoring function is relatively simple compared to some sophisticated methodologies that are available, the calculations are performed extremely rapidly. It should also be understood that the objective is not to calculate the true binding energy per se for each peptide docked in the binding groove of a selected MHC Class II protein. The underlying objective is to obtain comparative binding energy data as an aid to predicting the location of T-cell epitopes based on the primary structure (i.e. amino acid sequence) of a selected protein. A relatively high binding energy or a binding energy above a selected threshold value would suggest the presence of a T-cell epitope in the ligand. The ligand may then be subjected to at least one round of amino-acid substitution and the binding energy recalculated. Due to the rapid nature of the calculations, these manipulations of the peptide sequence can be performed interactively within the program's user interface on cost-effectively available computer hardware. Major investment in computer hardware is thus not required. It would be apparent to one skilled in the art that other available software could be used for the same purposes. In particular, more sophisticated software which is capable of docking ligands into protein binding-sites may be used in conjunction with energy minimization. Examples of docking software are: DOCK (Kuntz et al., J. Mol. Biol., 161:269-288 (1982)), LUDI (Böhm, H. J., J. Comput Aided Mol. Des., 8:623-632 (1994)) and FLEXX (Rarey M., et al., ISMB, 3:300-308 (1995)). Examples of molecular modeling and manipulation software include: AMBER (Tripos) and CHARMm (Molecular Simulations Inc.). The use of these computational methods would severely limit the throughput of the method of this invention due to the lengths of processing time required to make the necessary calculations. However, it is feasible that such methods could be used as a ‘secondary screen’ to obtain more accurate calculations of binding energy for peptides which are found to be ‘positive binders’ via the method of the present invention.

The limitation of processing time for sophisticated molecular mechanic or molecular dynamic calculations is one which is defined both by the design of the software which makes these calculations and the current technology limitations of computer hardware. It may be anticipated that, in the future, with the writing of more efficient code and the continuing increases in speed of computer processors, it may become feasible to make such calculations within a more manageable time-frame.

Further information on energy functions applied to macromolecules and consideration of the various interactions that take place within a folded protein structure can be found in: Brooks, B. R., et al., J. Comput. Chem., 4:187-217 (1983) and further information concerning general protein-ligand interactions can be found in: Dauber-Osguthorpe et al., Proteins4(1):31-47(1988), which are incorporated herein by reference in their entirety. Useful background information can also be found, for example, in Fasman, G. D., ed., Prediction of Protein Structure and the Principles of Protein Conformation, Plenum Press, New York, ISBN: 0-306 4313-9.

### EXAMPLE 2

Method of Mapping Epitopes in Bryodin 1 Using Naïve Human T-Cell Proliferation Assays:

The interaction between MHC, peptide and T-cell receptor (TCR) provides the structural basis for the antigen specificity of T-cell recognition. T-cell proliferation assays test the binding of peptides to MHC and the recognition of MHC/peptide complexes by the TCR. In vitro T-cell proliferation assays of the present example, involve the stimulation of peripheral blood mononuclear cells (PBMCs), containing antigen presenting cells (APCs) and T-cells. Stimulation is conducted in vitro using synthetic peptide antigens, and in some experiments whole protein antigen. Stimulated T-cell proliferation is measured using ³H-thymidine (³H-Thy) and the presence of incorporated ³H-Thy assessed using scintillation counting of washed fixed cells.

Buffy coats from human blood stored for less than 12 hours were obtained from the National Blood Service (Addenbrooks Hospital, Cambridge, UK). Ficoll-paque was obtained from Amersham Pharmacia Biotech (Amersham, UK). Serum free AIM V media for the culture of primary human lymphocytes and containing L-glutamine, 50 μg/ml streptomycin, 10 μg/ml gentomycin and 0.1% human serum albumin was from Gibco-BRL (Paisley, UK). Synthetic peptides were obtained from Eurosequence (Groningen, The Netherlands) and Babraham Technix (Cambridge, UK).

Erythrocytes and leukocytes were separated from plasma and platelets by gentle centrifugation of buffy coats. The top phase (containing plasma and platelets) was removed and discarded. Erythrocytes and leukocytes were diluted 1:1 in phosphate buffered saline (PBS) before layering onto 15 ml ficoll-paque (Amersham Pharmacia, Amersham UK). Centrifugation was done according to the manufacturers recommended conditions and PBMCs were harvested from the serum+PBS/ficoll paque interface. PBMCs were mixed with PBS (1:1) and collected by centrifugation. The supernatant was removed and discarded and the PBMC pellet resuspended in 50 ml PBS. Cells were again pelleted by centrifugation and the PBS supernatant discarded. Cells were resuspended using 50 ml AIM V media and at this point counted and viability assessed using trypan blue dye exclusion. Cells were again collected by centrifugation and the supernatant discarded. Cells were resuspended for cryogenic storage at a density of 3×10⁷ per ml. The storage medium was 90% (v/v) heat inactivated AB human serum (Sigma, Poole, UK) and 10% (v/v) DMSO (Sigma, Poole, UK). Cells were transferred to a regulated freezing container (Sigma) and placed at −70° C. overnight. When required for use, cells were thawed rapidly in a water bath at 37° C. before transferring to 10 ml pre-warmed AIM V medium.

PBMC were stimulated with protein and peptide antigens in a 96 well flat bottom plate at a density of 2×10⁵ PBMC per well. PBMC were incubated for 7 days at 37° C. before pulsing with ³H-Thy (Amersham-Phamacia, Amersham, UK). For the present study, synthetic peptides (15 mers) that overlapped by 12 amino acids were generated that spanned the entire sequence of bryodin 1. Peptide identification numbers (ID#) and sequences are given in FIG. 2. Each peptide was screened individually against PBMC's isolated from 21 naïve donors. Two control peptides that have previously been shown to be immunogenic and a potent non-recall antigen KLH were used in each donor assay.

The control antigens used in this study were as below:

| Peptide | Sequence |
|---|---|
| C-32 | Biotin-PKYVKQNTLKLAT |
| | Flu haemagglutinin 307-319 |
| C-49 | KVVDQIKKISKPVQH |
| | Chlamydia HSP 60 peptide |
| KLH | Whole protein from Keyhole |
| | Limpet Hemocyanin. |

Peptides were dissolved in DMSO to a final concentration of 10 mM, these stock solutions were then diluted 1/500 in AIM V media (final concentration 20 μM). Peptides were added to a flat bottom 96 well plate to give a final concentration of 2 and 20 μM in a 100 μl. The viability of thawed PBMC's was assessed by trypan blue dye exclusion, cells were then resuspended at a density of 2×10⁶ cells/ml, and 100 μl (2×10⁵ PBMC/well) was transferred to each well containing peptides. Triplicate well cultures were assayed at each peptide concentration. Plates were incubated for 7 days in a humidified atmosphere of 5% CO₂ at 37° C. Cells were pulsed for 18-21 hours with 1 μCi ³H-Thy/well before harvesting onto filter mats. CPM values were determined using a Wallac microplate beta top plate counter (Perkin Elmer). Results were expressed as stimulation indices, derived by division of the proliferation score (e.g. counts per minute of radioactivity) measured to the test peptide by the score measured in cells not contacted with a test peptide.

The present studies have uncovered some 32 peptide sequences able to evoke a significant proliferative response (i.e. SI>2.0) in. T-cells derived from at least one donor. Within this set of peptides, a further sub-set of peptides have been identified which evoke a significant proliferative response in 2 or more individual donor samples and for some of these responses the magnitude of response has indeed been significantly higher than SI=2.0. Mapping T cell epitopes in the bryodin 1 sequence using the T cell proliferation assay resulted in the identification of five major immunogenic regions encompassed by peptide ID#16-18, 30, 38-41, 46-50 and 60-64. For each of these peptides, PBMCs prepared from 2 or more donor samples showed a stimulation index >2.0. FIG. 5 panels A-E show representative histograms of SI responses to individual peptides in selected PBMC donor samples. Collectively the panels have been selected to demonstrate examples of positive responses to peptides from each of the epitope regions R1- R5. The tissue types for all PBMC samples were assayed using a commercially available reagent system (Dynal, Wirral, UK). Assays were conducted in accordance with the suppliers recommended protocols and standard ancillary reagents and agarose electrophoresis systems. Allotypic coverage for DRB1 alleles was 70% in the 20 donors tested. Of the 21 different PBMC donor preparations 10 were reactive to peptides encompassed within epitope regions R1- R5. The allotypic specificities of each of the ve donor samples is given in TABLE 1.

TABLE 1
MHC Allotypes of responsive donor samples

| Donor # | MHC Allotype |
|---|---|
| 4 | DRB1*03, DRB1*04, DRB3, DRB4*01 |
| 5 | DRB1*07, DRB1*09, DRB4*01 |
| 6 | DRB1*13, DRB1*15, DRB3, DRB5 |
| 9 | DRB1*04, DRB1*12, DRB3, DRB4*01 |
| 12 | DRB1*07, DRB1*13, DRB3, DRB4*01 |
| 14 | DRB1*04, DRB4*01 |
| 15 | DRB1*03, DRB1*14, DRB3 |
| 17 | DRB1*12, DRB1*15, DRB3, DRB5 |
| 19 | DRB1*03, DRB1*07, DRB3, DRB4*01 |
| 21 | DRB1*08, DRB1*14, DRB3 |

### EXAMPLE 3

Design of Modified Sequences with Improved Immunogenicity Profiles:

The method of EXAMPLE 1 was used in an analysis of the epitope regions R1, R2, R3, R4 and R5. The system enables prediction of the particular MHC ligands encompassed within the biologically detected epitope regions and provides a “score” with respect to the ability of a given MHC class II ligand to interact with a particular MHC allotype. The allotypic restriction pattern for the MHC ligands can be depicted using the allotypic restriction chart displays as provided for each of the epitope regions R1-R5 in the accompanying FIGS. 6-10.

The analysis was extended to consideration of sequence modifications within each of the epitopes R1- R5. The sequence variants were tested for continued ability bind MHC class II and their binding scores where these remained. Multiple amino acid substitutions were defined which achieved elimination of MHC class II binding with the majority of MHC allotypes tested. The particular substitutions identified were further tested for their ability to be accommodated within the structural model of the bryodin molecule. Designed mutations on the selected residues of the wild type sequence were checked for steric clashes, hydrogen bonding formation, hydrophobic interactions and its general accommodation in the structure. Substitutions that gave rise to steric clashes were dismissed. Substitutions that were accommodated when the side chain was adopting a similar configuration (rotamer) to the original residue was considered acceptable. If more than one substitutions fulfilled these criteria, residues that potentially form hydrogen bonds with neighbouring side chains or backbone atoms, and/or form favourable hydrophobic contacts or other associations were preferred. The above procedure was performed interactively using Swiss Prot Deep View v3.7 [Guex, N. and Peitsch, M. C. (1997) Electrophoresis 18: 2714-2723]. This process resulted in a preferred substitution set for each of the epitope regions R1- R5. The substitution sets were compiled to produce the structure depicted in FORMULA 1. All substitutions were confirmed to result in removal of the MHC class II ligands within each of the epitope regions R1- R5. Substitutions featuring alternative amino acid residues in addition to the most preferred substitutions are given with the FORMULA 1.

For epitope region R3 alternative substitution sets were designed enabling the option of leaving leucine 115 in the wild-type configuration. This residue it thought to be structurally important forming part of the substrate binding cleft for the bryodin 1 enzyme. A preferred set of substitutions involving L115 comprises the changes L₁₁₅A, I₁₂₂A, I₁₂₆A L₁₃₀A, L₁₃₃F and I₁₃₇A. An alternative set of substitutions which maintain L₁₁₅ comprise A₁₁₈T, G₁₂₀H, K₁₂₁S and R₁₂₃T. These changes would be made in alternative to the dual changes L₁₁₅A and I₁₂₂A.

1. A modified molecule having the biological activity of bryodin 1 and being substantially non-immunogenic or less immunogenic than any non-modified molecule having the same biological activity in an individual when used in vivo, wherein the said loss of immunogenicity is achieved by removing one or more T-cell epitopes derived from the originally non-modified molecule and said T-cell epitopes are MHC class II ligands or peptide sequences which show the ability to stimulate or bind T-cells via presentation on class II.

2. A byrodin 1 molecule of claim 1, wherein the removing of said T-cell epitopes are achieved by replacing 1-9 amino acid residues.

3. (canceled)

4. A byrodin 1 molecule of claim 1, wherein said T-cell epitopes are located within the strings of contiguous amino acid residues termed as R1- R5 encompassing residues 46-66; 88-102; 112-135; 136-162 and 178-204 of the wild-type byrodin 1 sequence (SEQ ID NO: 1).

5-16. (canceled)

17. A polypeptide having the biological activity of human bryodin 1, the polypeptide comprising the amino acid residue sequence of wild-type human bryodin 1, SEQ ID NO: 1, and including at least one amino acid residue substitution in an epitope region of SEQ ID NO: 1, the amino acid residue substitution rendering the polypeptide less immunogenic to a human than wild-type human bryodin 1.

18. The polypeptide of claim 17 wherein the at least one amino acid residue substitution comprises one to nine amino acid residue substitutions in SEQ ID NO: 1.

19. The polypeptide of claim 17 wherein the at least one amino acid residue substitution is a substitution in at least one epitope region of SEQ ID NO: 1 selected from the group consisting of amino acid residues 46-66 of SEQ ID NO: 1, amino acid residues 88-102 of SEQ ID NO: 1, amino acid residues 112-135 of SEQ ID NO: 1, amino acid residues 136-162 of SEQ ID NO: 1, and amino acid residues 178-204 of SEQ ID NO: 1.

20. The polypeptide of claim 19 wherein the at least one amino acid residue substitution comprises one to nine amino acid residue substitutions in SEQ ID NO: 1.

21. A polypeptide comprising the amino acid residue sequence of SEQ ID NO: 7:
DVSFRLSGATTTSYGVFIKNLREALPYERKVYNIPLLRSSISGSGRYX¹X²LX³LTX⁴X⁵ADETX⁶SVAX⁷DX⁸TNVYIMGYLAGDVSYFFNEASATEAAKX⁹X¹⁰FKDAKKKX¹¹TLPYSGNYERX¹²QTX¹³AX¹⁴X¹⁵X¹⁶X¹⁷ENX¹⁸PLGX¹⁹PAX²⁰DSAX²¹TTX²²YX²³X²⁴TASSAASAX²⁵X²⁶X²⁷X²⁸IQSTAESARYKFIEQQIGKRVDKTFLP SLATX29SX³⁰ENNWSAX³¹SX³²QX³³QX³⁴ASTNNGQFESPVVLIDGNNQRVSITNASARVVTSNIALLLNRN NIAAIGEDISMTLIGFEHGLYGI (SEQ ID NO: 7)
wherein
X¹ is A, G or P; X² is M, A, G, P or I; X³ is A, G or P; X⁴ is P or Y;
X⁵ is T or S; X⁶ is P; X⁷ is A, P or G; X⁸ is A, P or G;
X⁹ is A, P, G, H, D, E, N, Q, K, R, S or T; X¹⁰ is A, P or G; X¹¹ is A, P or G;
X¹² is A, P, S, T, H or K; X¹³ is T; X¹⁴ is H; X¹⁵ is S;
X¹⁶ is A, S, T, P, N, D, E, G, H, K or Q; X¹⁷ is T; or P;
X¹⁹ is A, I, F, G, M, P, V, W or Y; X²⁰ is F, P or W; X²¹ is A, P or G;
X²² is G, A or P; X²³ is G, A or P; X²⁴ is A, P or G; X²⁵ is A, P, G, S or T;
X²⁶ is A, I, M, S, T, P or G; X²⁷ is A, G or P;
X²⁸ is S, A, G, P, T, H, D, N, Q, K or R;
X²⁹ is T, A, G, S, P, H, K, R, D, E, N or Q;
X³⁰ is A, G, S, T, P, K, R, H, D, E, N or Q; X³¹ is Q;
X³² is H, D, E, F, L, N, P, S, W or Y;
X³³ is T, A, G, P, D, E, H, K, R, N, Q, S or T; and X³⁴ is D;
with the proviso that the variable amino acid residues X¹ through X³⁴ do not simultaneously have the following combination of identities:
X¹=T, X²=L, X³═H, X⁴═N, X⁵═Y, X⁶═I, X⁷═V, X⁸═V, X⁹═F, X¹⁰═V, X¹¹═V, X¹²=L, X¹³=A, X¹⁴=G, X¹⁵═K, X¹⁶═I, X¹⁷═R, X¹⁸═I, X¹⁹=L, X²⁰=L, X²¹═I, X²²=L, X²³═Y, X²⁴═Y, X²⁵=L, X²⁶=L, X²⁷═V, X²⁸=L, X²⁹═I, X³⁰=L, X³¹=L, X³²═K, X³³═I, and X³⁴═I.

22. The polypeptide of claim 21 wherein X¹ is A, X² is M, X3 is A, X⁴ is P, X⁵ is T, X⁶ is P, X⁷ is A, X⁸ is A, X⁹ is A, X¹⁰ is A, X¹¹ is A, X¹² is A, X¹³ is T, X¹⁴ is H, X¹⁵ is S, X¹⁶ is A, X¹⁷ is T, X¹⁸ is A, X¹⁹ is A, X²⁰ is F, X²¹ is A, X²² is G, X²³ is G, X²⁴ is A, X²⁵ is A, X²⁶ is A, X²⁷ is A, X²⁸ is S, X²⁹ is T, X³⁰ is A, X³¹ is Q, X³² is H, X³³ is T and X³⁴ is D.

23. The polypeptide of claim 21 wherein the polypeptide exhibits, in a biological assay of induced cellular proliferation of human T-cells, a stimulation index (SI) having a value less than 2 and less than the SI of wild-type human bryodin 1, when the polypeptide and wild-type human bryodin 1 are tested in parallel using cells from the same donor, and wherein the SI is determined as the value of cellular proliferation obtained from T-cells stimulated with the polypeptide or bryodin 1, divided by the value of cellular proliferation determined in control T-cells not exposed to the polypeptide or bryodin 1, respectively.

24. A pharmaceutical composition comprising a polypeptide of claim 17 together with a material selected from the group consisting of a carrier, a diluent, and an excipient.

25. A pharmaceutical composition comprising a polypeptide of claim 21 together with a material selected from the group consisting of a carrier, a diluent, and an excipient.

26. An isolated polypeptide consisting of an amino acid residue sequence selected from the group consisting of SEQ ID NO: 2, SEQ ID NO: 3, SEQ ID NO: 4, SEQ ID NO: 5, and SEQ ID NO: 6.

27. An isolated polypeptide consisting of any one of the amino acid residues sequences depicted in FIG. 1.

28. An isolated polypeptide consisting of any one of the amino acid residues sequences depicted in FIG. 2.

29. An isolated deoxyribonucleic acid encoding a polypeptide of claim 17.

30. An isolated deoxyribonucleic acid encoding a polypeptide of claim 21.


SEQUENCE LISTING (183 sequences)

SEQ ID NO: 1 - protein, 267 aa, Homo sapiens
DVSFRLSGATTTSYGVFIKNLREALPYERKVYNIPLLRSSISGSGRYTLLHLTNYADETISVAVDVTNVYIMGYLAGDVSYFFNEASATEAAKFVFKDAKKKVTLPYSGNYERLQTAAGKIRENIPLGLPALDSAITTLYYYTASSAASALLVLIQSTAESARYKFIEQQIGKRVDKTFLPSLATISLENNWSALSKQIQIASTNNGQFESPVVLIDGNNQRVSITNASARVVTSNIALLLNRNNIAAIGEDISMTLIGFEHGLYGI

SEQ ID NO: 2 - protein, 21 aa, Homo sapiens
RYTLLHLTNYADETISVAVDV

SEQ ID NO: 3 - protein, 15 aa, Homo sapiens
ATEAAKFVFKDAKKK

SEQ ID NO: 4 - protein, 24 aa, Homo sapiens
ERLQTAAGKIRENIPLGLPALDSA

SEQ ID NO: 5 - protein, 27 aa, Homo sapiens
ITTLYYYTASSAASALLVLIQSTAESA

SEQ ID NO: 6 - protein, 21 aa, Homo sapiens
ATISLENNWSALSKQIQIAST

SEQ ID NO: 7 - protein, 267 aa, Artificial Sequence
  Modified byrodin 1 protein
DVSFRLSGATTTSYGVFIKNLREALPYERKVYNIPLLRSSISGSGRYXXLXLTXXADETXSVAXDXTNVYIMGYLAGDVSYFFNEASATEAAKXXFKDAKKKXTLPYSGNYERXQTXAXXXXENXPLGXPAXDSAXTTXYXXTASSAASAXXXXIQSTAESARYKFIEQQIGKRVDKTFLPSLATXSXENNWSAXSXQXQXASTNNGQFESPVVLIDGNNQRVSITNASARVVTSNIALLLNRNNIAAIGEDISMTLIGFEHGLYGI

SEQ ID NO: 8 - protein, 13 aa, Artificial Sequence
  Flu protein fragment
PKYVKQNTLKLAT

SEQ ID NO: 9 - protein, 15 aa, Artificial Sequence
  Chlamydia peptide
KVVDQIKKISKPVQH

SEQ ID NO: 10 - protein, 13 aa, Artificial Sequence
  Fragments of Bryodin 1
VSFRLSGATTTSY

SEQ ID NO: 11 - protein, 13 aa, Artificial Sequence
  Fragments of Bryodin 1
FRLSGATTTSYGV

SEQ ID NO: 12 - protein, 13 aa, Artificial Sequence
  Fragments of Bryodin 1
TSYGVFIKNLREA

SEQ ID NO: 13 - protein, 13 aa, Artificial Sequence
  Fragments of Bryodin 1
YGVFIKNLREALP

SEQ ID NO: 14 - protein, 13 aa, Artificial Sequence
  Fragments of Bryodin 1
GVFIKNLREALPY

SEQ ID NO: 15 - protein, 13 aa, Artificial Sequence
  Fragments of Bryodin 1
VFIKNLREALPYE

SEQ ID NO: 16 - protein, 13 aa, Artificial Sequence
  Fragments of Bryodin 1
KNLREALPYERKV

SEQ ID NO: 17 - protein, 13 aa, Artificial Sequence
  Fragments of Bryodin 1
EALPYERKVYNIP

SEQ ID NO: 18 - protein, 13 aa, Artificial Sequence
  Fragments of Bryodin 1
LPYERKVYNIPLL

SEQ ID NO: 19 - protein, 13 aa, Artificial Sequence
  Fragments of Bryodin 1
RKVYNIPLLRSSI

SEQ ID NO: 20 - protein, 13 aa, Artificial Sequence
  Fragments of Bryodin 1
KVYNIPLLRSSIS

SEQ ID NO: 21 - protein, 13 aa, Artificial Sequence
  Fragments of Bryodin 1
YNIPLLRSSISGS

SEQ ID NO: 22 - protein, 13 aa, Artificial Sequence
  Fragments of Bryodin 1
IPLLRSSISGSGR

SEQ ID NO: 23 - protein, 13 aa, Artificial Sequence
  Fragments of Bryodin 1
PLLRSSISGSGRY

SEQ ID NO: 24 - protein, 13 aa, Artificial Sequence
  Fragments of Bryodin 1
SSISGSGRYTLLH

SEQ ID NO: 25 - protein, 13 aa, Artificial Sequence
  Fragments of Bryodin 1
GRYTLLHLTNYAD

SEQ ID NO: 26 - protein, 13 aa, Artificial Sequence
  Fragments of Bryodin 1
YTLLHLTNYADET

SEQ ID NO: 27 - protein, 13 aa, Artificial Sequence
  Fragments of Bryodin 1
TLLHLTNYADETI

SEQ ID NO: 28 - protein, 13 aa, Artificial Sequence
  Fragments of Bryodin 1
LHLTNYADETISV

SEQ ID NO: 29 - protein, 13 aa, Artificial Sequence
  Fragments of Bryodin 1
TNYADETISVAVD

SEQ ID NO: 30 - protein, 13 aa, Artificial Sequence
  Fragments of Bryodin 1
ETISVAVDVTNVY

SEQ ID NO: 31 - protein, 13 aa, Artificial Sequence
  Fragments of Bryodin 1
ISVAVDVTNVYIM

SEQ ID NO: 32 - protein, 13 aa, Artificial Sequence
  Fragments of Bryodin 1
VAVDVTNVYIMGY

SEQ ID NO: 33 - protein, 13 aa, Artificial Sequence
  Fragments of Bryodin 1
VDVTNVYIMGYLA

SEQ ID NO: 34 - protein, 13 aa, Artificial Sequence
  Fragments of Bryodin 1
TNVYIMGYLAGDV

SEQ ID NO: 35 - protein, 13 aa, Artificial Sequence
  Fragments of Bryodin 1
NVYIMGYLAGDVS

SEQ ID NO: 36 - protein, 13 aa, Artificial Sequence
  Fragments of Bryodin 1
VYIMGYLAGDVSY

SEQ ID NO: 37 - protein, 13 aa, Artificial Sequence
  Fragments of Bryodin 1
YIMGYLAGDVSYF

SEQ ID NO: 38 - protein, 13 aa, Artificial Sequence
  Fragments of Bryodin 1
MGYLAGDVSYFFN

SEQ ID NO: 39 - protein, 13 aa, Artificial Sequence
  Fragments of Bryodin 1
GYLAGDVSYFFNE

SEQ ID NO: 40 - protein, 13 aa, Artificial Sequence
  Fragments of Bryodin 1
GDVSYFFNEASAT

SEQ ID NO: 41 - protein, 13 aa, Artificial Sequence
  Fragments of Bryodin 1
VSYFFNEASATEA

SEQ ID NO: 42 - protein, 13 aa, Artificial Sequence
  Fragments of Bryodin 1
SYFFNEASATEAA

SEQ ID NO: 43 - protein, 13 aa, Artificial Sequence
  Fragments of Bryodin 1
YFFNEASATEAAK

SEQ ID NO: 44 - protein, 13 aa, Artificial Sequence
  Fragments of Bryodin 1
AKFVFKDAKKKVT

SEQ ID NO: 45 - protein, 13 aa, Artificial Sequence
  Fragments of Bryodin 1
KFVFKDAKKKVTL

SEQ ID NO: 46 - protein, 13 aa, Artificial Sequence
  Fragments of Bryodin 1
FVFKDAKKKVTLP

SEQ ID NO: 47 - protein, 13 aa, Artificial Sequence
  Fragments of Bryodin 1
KKVTLPYSGNYER

SEQ ID NO: 48 - protein, 13 aa, Artificial Sequence
  Fragments of Bryodin 1
VTLPYSGNYERLQ

SEQ ID NO: 49 - protein, 13 aa, Artificial Sequence
  Fragments of Bryodin 1
LPYSGNYERLQTA

SEQ ID NO: 50 - protein, 13 aa, Artificial Sequence
  Fragments of Bryodin 1
GNYERLQTAAGKI

SEQ ID NO: 51 - protein, 13 aa, Artificial Sequence
  Fragments of Bryodin 1
ERLQTAAGKIREN

SEQ ID NO: 52 - protein, 13 aa, Artificial Sequence
  Fragments of Bryodin 1
GKIRENIPLGLPA

SEQ ID NO: 53 - protein, 13 aa, Artificial Sequence
  Fragments of Bryodin 1
ENIPLGLPALDSA

SEQ ID NO: 54 - protein, 13 aa, Artificial Sequence
  Fragments of Bryodin 1
IPLGLPALDSAIT

SEQ ID NO: 55 - protein, 13 aa, Artificial Sequence
  Fragments of Bryodin 1
LGLPALDSAITTL

SEQ ID NO: 56 - protein, 13 aa, Artificial Sequence
  Fragments of Bryodin 1
PALDSAITTLYYY

SEQ ID NO: 57 - protein, 13 aa, Artificial Sequence
  Fragments of Bryodin 1
SAITTLYYYTASS

SEQ ID NO: 58 - protein, 13 aa, Artificial Sequence
  Fragments of Bryodin 1
TTLYYYTASSAAS

SEQ ID NO: 59 - protein, 13 aa, Artificial Sequence
  Fragments of Bryodin 1
TLYYYTASSAASA

SEQ ID NO: 60 - protein, 13 aa, Artificial Sequence
  Fragments of Bryodin 1
LYYYTASSAASAL

SEQ ID NO: 61 - protein, 13 aa, Artificial Sequence
  Fragments of Bryodin 1
YYYTASSAASALL

SEQ ID NO: 62 - protein, 13 aa, Artificial Sequence
  Fragments of Bryodin 1
SALLVLIQSTAES

SEQ ID NO: 63 - protein, 13 aa, Artificial Sequence
  Fragments of Bryodin 1
ALLVLIQSTAESA

SEQ ID NO: 64 - protein, 13 aa, Artificial Sequence
  Fragments of Bryodin 1
LLVLIQSTAESAR

SEQ ID NO: 65 - protein, 13 aa, Artificial Sequence
  Fragments of Bryodin 1
LVLIQSTAESARY

SEQ ID NO: 66 - protein, 13 aa, Artificial Sequence
  Fragments of Bryodin 1
VLIQSTAESARYK

SEQ ID NO: 67 - protein, 13 aa, Artificial Sequence
  Fragments of Bryodin 1
ARYKFIEQQIGKR

SEQ ID NO: 68 - protein, 13 aa, Artificial Sequence
  Fragments of Bryodin 1
YKFIEQQIGKRVD

SEQ ID NO: 69 - protein, 13 aa, Artificial Sequence
  Fragments of Bryodin 1
KFIEQQIGKRVDK

SEQ ID NO: 70 - protein, 13 aa, Artificial Sequence
  Fragments of Bryodin 1
QQIGKRVDKTFLP

SEQ ID NO: 71 - protein, 13 aa, Artificial Sequence
  Fragments of Bryodin 1
KRVDKTFLPSLAT

SEQ ID NO: 72 - protein, 13 aa, Artificial Sequence
  Fragments of Bryodin 1
KTFLPSLATISLE

SEQ ID NO: 73 - protein, 13 aa, Artificial Sequence
  Fragments of Bryodin 1
TFLPSLATISLEN

SEQ ID NO: 74 - protein, 13 aa, Artificial Sequence
  Fragments of Bryodin 1
PSLATISLENNWS

SEQ ID NO: 75 - protein, 13 aa, Artificial Sequence
  Fragments of Bryodin 1
ATISLENNWSALS

SEQ ID NO: 76 - protein, 13 aa, Artificial Sequence
  Fragments of Bryodin 1
ISLENNWSALSKQ

SEQ ID NO: 77 - protein, 13 aa, Artificial Sequence
  Fragments of Bryodin 1
NNWSALSKQIQIA

SEQ ID NO: 78 - protein, 13 aa, Artificial Sequence
  Fragments of Bryodin 1
SALSKQIQIASTN

SEQ ID NO: 79 - protein, 13 aa, Artificial Sequence
  Fragments of Bryodin 1
KQIQIASTNNGQF

SEQ ID NO: 80 - protein, 13 aa, Artificial Sequence
  Fragments of Bryodin 1
IQIASTNNGQFES

SEQ ID NO: 81 - protein, 13 aa, Artificial Sequence
  Fragments of Bryodin 1
GQFESPVVLIDGN

SEQ ID NO: 82 - protein, 13 aa, Artificial Sequence
  Fragments of Bryodin 1
SPVVLIDGNNQRV

SEQ ID NO: 83 - protein, 13 aa, Artificial Sequence
  Fragments of Bryodin 1
PVVLIDGNNQRVS

SEQ ID NO: 84 - protein, 13 aa, Artificial Sequence
  Fragments of Bryodin 1
VVLIDGNNQRVSI

SEQ ID NO: 85 - protein, 13 aa, Artificial Sequence
  Fragments of Bryodin 1
VLIDGNNQRVSIT

SEQ ID NO: 86 - protein, 13 aa, Artificial Sequence
  Fragments of Bryodin 1
QRVSITNASARVV

SEQ ID NO: 87 - protein, 13 aa, Artificial Sequence
  Fragments of Bryodin 1
VSITNASARVVTS

SEQ ID NO: 88 - protein, 13 aa, Artificial Sequence
  Fragments of Bryodin 1
ARVVTSNIALLLN

SEQ ID NO: 89 - protein, 13 aa, Artificial Sequence
  Fragments of Bryodin 1
RVVTSNIALLLNR

SEQ ID NO: 90 - protein, 13 aa, Artificial Sequence
  Fragments of Bryodin 1
SNIALLLNRNNIA

SEQ ID NO: 91 - protein, 13 aa, Artificial Sequence
  Fragments of Bryodin 1
IALLLNRNNIAAI

SEQ ID NO: 92 - protein, 13 aa, Artificial Sequence
  Fragments of Bryodin 1
ALLLNRNNIAAIG

SEQ ID NO: 93 - protein, 13 aa, Artificial Sequence
  Fragments of Bryodin 1
LLLNRNNIAAIGE

SEQ ID NO: 94 - protein, 13 aa, Artificial Sequence
  Fragments of Bryodin 1
NNIAAIGEDISMT

SEQ ID NO: 95 - protein, 13 aa, Artificial Sequence
  Fragments of Bryodin 1
AAIGEDISMTLIG

SEQ ID NO: 96 - protein, 13 aa, Artificial Sequence
  Fragments of Bryodin 1
EDISMTLIGFEHG

SEQ ID NO: 97 - protein, 13 aa, Artificial Sequence
  Fragments of Bryodin 1
ISMTLIGFEHGLY

SEQ ID NO: 98 - protein, 13 aa, Artificial Sequence
  Fragments of Bryodin 1
MTLIGFEHGLYGI

SEQ ID NO: 99 - protein, 15 aa, Artificial Sequence
  Fragments of Bryodin 1
DVSFRLSGATTTSYG

SEQ ID NO: 100 - protein, 15 aa, Artificial Sequence
  Fragments of Bryodin 1
FRLSGATTTSYGVFI

SEQ ID NO: 101 - protein, 15 aa, Artificial Sequence
  Fragments of Bryodin 1
SGATTTSYGVFIKNL

SEQ ID NO: 102 - protein, 15 aa, Artificial Sequence
  Fragments of Bryodin 1
TTTSYGVFIKNLREA

SEQ ID NO: 103 - protein, 15 aa, Artificial Sequence
  Fragments of Bryodin 1
SYGVFIKNLREALPY

SEQ ID NO: 104 - protein, 15 aa, Artificial Sequence
  Fragments of Bryodin 1
VFIKNLREALPYERK

SEQ ID NO: 105 - protein, 15 aa, Artificial Sequence
  Fragments of Bryodin 1
KNLREALPYERKVYN

SEQ ID NO: 106 - protein, 15 aa, Artificial Sequence
  Fragments of Bryodin 1
REALPYERKVYNIPL

SEQ ID NO: 107 - protein, 15 aa, Artificial Sequence
  Fragments of Bryodin 1
LPYERKVYNIPLLRS

SEQ ID NO: 108 - protein, 15 aa, Artificial Sequence
  Fragments of Bryodin 1
ERKVYNIPLLRSSIS

SEQ ID NO: 109 - protein, 15 aa, Artificial Sequence
  Fragments of Bryodin 1
VYNIPLLRSSISGSG

SEQ ID NO: 110 - protein, 15 aa, Artificial Sequence
  Fragments of Bryodin 1
IPLLRSSISGSGRYT

SEQ ID NO: 111 - protein, 15 aa, Artificial Sequence
  Fragments of Bryodin 1
LRSSISGSGRYTLLH

SEQ ID NO: 112 - protein, 15 aa, Artificial Sequence
  Fragments of Bryodin 1
SISGSGRYTLLHLTN

SEQ ID NO: 113 - protein, 15 aa, Artificial Sequence
  Fragments of Bryodin 1
GSGRYTLLHLTNYAD

SEQ ID NO: 114 - protein, 15 aa, Artificial Sequence
  Fragments of Bryodin 1
RYTLLHLTNYADETI

SEQ ID NO: 115 - protein, 15 aa, Artificial Sequence
  Fragments of Bryodin 1
LLHLTNYADETISVA

SEQ ID NO: 116 - protein, 15 aa, Artificial Sequence
  Fragments of Bryodin 1
LTNYADETISVAVDV

SEQ ID NO: 117 - protein, 15 aa, Artificial Sequence
  Fragments of Bryodin 1
YADETISVAVDVTNV

SEQ ID NO: 118 - protein, 15 aa, Artificial Sequence
  Fragments of Bryodin 1
ETISVAVDVTNVYIM

SEQ ID NO: 119 - protein, 15 aa, Artificial Sequence
  Fragments of Bryodin 1
SVAVDVTNVYIMGYL

SEQ ID NO: 120 - protein, 15 aa, Artificial Sequence
  Fragments of Bryodin 1
VDVTNVYIMGYLAGD

SEQ ID NO: 121 - protein, 15 aa, Artificial Sequence
  Fragments of Bryodin 1
TNVYIMGYLAGDVSY

SEQ ID NO: 122 - protein, 15 aa, Artificial Sequence
  Fragments of Bryodin 1
YIMGYLAGDVSYFFN

SEQ ID NO: 123 - protein, 15 aa, Artificial Sequence
  Fragments of Bryodin 1
GYLAGDVSYFFNEAS

SEQ ID NO: 124 - protein, 15 aa, Artificial Sequence
  Fragments of Bryodin 1
AGDVSYFFNEASATE

SEQ ID NO: 125 - protein, 15 aa, Artificial Sequence
  Fragments of Bryodin 1
VSYFFNEASATEAAK

SEQ ID NO: 126 - protein, 15 aa, Artificial Sequence
  Fragments of Bryodin 1
FFNEASATEAAKFVF

SEQ ID NO: 127 - protein, 15 aa, Artificial Sequence
  Fragments of Bryodin 1
EASATEAAKFVFKDA

SEQ ID NO: 128 - protein, 15 aa, Artificial Sequence
  Fragments of Bryodin 1
ATEAAKFVFKDAKKK

SEQ ID NO: 129 - protein, 15 aa, Artificial Sequence
  Fragments of Bryodin 1
AAKFVFKDAKKKVTL

SEQ ID NO: 130 - protein, 15 aa, Artificial Sequence
  Fragments of Bryodin 1
FVFKDAKKKVTLPYS

SEQ ID NO: 131 - protein, 15 aa, Artificial Sequence
  Fragments of Bryodin 1
KDAKKKVTLPYSGNY

SEQ ID NO: 132 - protein, 15 aa, Artificial Sequence
  Fragments of Bryodin 1
KKKVTLPYSGNYERL

SEQ ID NO: 133 - protein, 15 aa, Artificial Sequence
  Fragments of Bryodin 1
VTLPYSGNYERLQTA

SEQ ID NO: 134 - protein, 15 aa, Artificial Sequence
  Fragments of Bryodin 1
PYSGNYERLQTAAGK

SEQ ID NO: 135 - protein, 15 aa, Artificial Sequence
  Fragments of Bryodin 1
GNYERLQTAAGKIRE

SEQ ID NO: 136 - protein, 15 aa, Artificial Sequence
  Fragments of Bryodin 1
ERLQTAAGKIRENIP

SEQ ID NO: 137 - protein, 15 aa, Artificial Sequence
  Fragments of Bryodin 1
QTAAGKIRENIPLGL

SEQ ID NO: 138 - protein, 15 aa, Artificial Sequence
  Fragments of Bryodin 1
AGKIRENIPLGLPAL

SEQ ID NO: 139 - protein, 15 aa, Artificial Sequence
  Fragments of Bryodin 1
IRENIPLGLPALDSA

SEQ ID NO: 140 - protein, 15 aa, Artificial Sequence
  Fragments of Bryodin 1
NIPLGLPALDSAITT

SEQ ID NO: 141 - protein, 15 aa, Artificial Sequence
  Fragments of Bryodin 1
LGLPALDSAITTLYY

SEQ ID NO: 142 - protein, 15 aa, Artificial Sequence
  Fragments of Bryodin 1
PALDSAITTLYYYTA

SEQ ID NO: 143 - protein, 15 aa, Artificial Sequence
  Fragments of Bryodin 1
DSAITTLYYYTASSA

SEQ ID NO: 144 - protein, 15 aa, Artificial Sequence
  Fragments of Bryodin 1
ITTLYYYTASSAASA

SEQ ID NO: 145 - protein, 15 aa, Artificial Sequence
  Fragments of Bryodin 1
LYYYTASSAASALLV

SEQ ID NO: 146 - protein, 15 aa, Artificial Sequence
  Fragments of Bryodin 1
YTASSAASALLVLIQ

SEQ ID NO: 147 - protein, 15 aa, Artificial Sequence
  Fragments of Bryodin 1
SSAASALLVLIQSTA

SEQ ID NO: 148 - protein, 15 aa, Artificial Sequence
  Fragments of Bryodin 1
ASALLVLIQSTAESA

SEQ ID NO: 149 - protein, 15 aa, Artificial Sequence
  Fragments of Bryodin 1
LLVLIQSTAESARYK

SEQ ID NO: 150 - protein, 15 aa, Artificial Sequence
  Fragments of Bryodin 1
LIQSTAESARYKFIE

SEQ ID NO: 151 - protein, 15 aa, Artificial Sequence
  Fragments of Bryodin 1
STAESARYKFIEQQI

SEQ ID NO: 152 - protein, 15 aa, Artificial Sequence
  Fragments of Bryodin 1
ESARYKFIEQQIGKR

SEQ ID NO: 153 - protein, 15 aa, Artificial Sequence
  Fragments of Bryodin 1
RYKFIEQQIGKRVDK

SEQ ID NO: 154 - protein, 15 aa, Artificial Sequence
  Fragments of Bryodin 1
FIEQQIGKRVDKTFL

SEQ ID NO: 155 - protein, 15 aa, Artificial Sequence
  Fragments of Bryodin 1
QQIGKRVDKTFLPSL

SEQ ID NO: 156 - protein, 15 aa, Artificial Sequence
  Fragments of Bryodin 1
GKRVDKTFLPSLATI

SEQ ID NO: 157 - protein, 15 aa, Artificial Sequence
  Fragments of Bryodin 1
VDKTFLPSLATISLE

SEQ ID NO: 158 - protein, 15 aa, Artificial Sequence
  Fragments of Bryodin 1
TFLPSLATISLENNW

SEQ ID NO: 159 - protein, 15 aa, Artificial Sequence
  Fragments of Bryodin 1
PSLATISLENNWSAL

SEQ ID NO: 160 - protein, 15 aa, Artificial Sequence
  Fragments of Bryodin 1
ATISLENNWSALSKQ

SEQ ID NO: 161 - protein, 15 aa, Artificial Sequence
  Fragments of Bryodin 1
SLENNWSALSKQIQI

SEQ ID NO: 162 - protein, 15 aa, Artificial Sequence
  Fragments of Bryodin 1
NNWSALSKQIQIAST

SEQ ID NO: 163 - protein, 15 aa, Artificial Sequence
  Fragments of Bryodin 1
SALSKQIQIASTNNG

SEQ ID NO: 164 - protein, 15 aa, Artificial Sequence
  Fragments of Bryodin 1
SKQIQIASTNNGQFE

SEQ ID NO: 165 - protein, 15 aa, Artificial Sequence
  Fragments of Bryodin 1
IQIASTNNGQFESPV

SEQ ID NO: 166 - protein, 15 aa, Artificial Sequence
  Fragments of Bryodin 1
ASTNNGQFESPVVLI

SEQ ID NO: 167 - protein, 15 aa, Artificial Sequence
  Fragments of Bryodin 1
NNGQFESPVVLIDGN

SEQ ID NO: 168 - protein, 15 aa, Artificial Sequence
  Fragments of Bryodin 1
QFESPVVLIDGNNQR

SEQ ID NO: 169 - protein, 15 aa, Artificial Sequence
  Fragments of Bryodin 1
SPVVLIDGNNQRVSI

SEQ ID NO: 170 - protein, 15 aa, Artificial Sequence
  Fragments of Bryodin 1
VLIDGNNQRVSITNA

SEQ ID NO: 171 - protein, 15 aa, Artificial Sequence
  Fragments of Bryodin 1
DGNNQRVSITNASAR

SEQ ID NO: 172 - protein, 15 aa, Artificial Sequence
  Fragments of Bryodin 1
NQRVSITNASARVVT

SEQ ID NO: 173 - protein, 15 aa, Artificial Sequence
  Fragments of Bryodin 1
VSITNASARVVTSNI

SEQ ID NO: 174 - protein, 15 aa, Artificial Sequence
  Fragments of Bryodin 1
TNASARVVTSNIALL

SEQ ID NO: 175 - protein, 15 aa, Artificial Sequence
  Fragments of Bryodin 1
SARVVTSNIALLLNR

SEQ ID NO: 176 - protein, 15 aa, Artificial Sequence
  Fragments of Bryodin 1
VVTSNIALLLNRNNI

SEQ ID NO: 177 - protein, 15 aa, Artificial Sequence
  Fragments of Bryodin 1
SNIALLLNRNNIAAI

SEQ ID NO: 178 - protein, 15 aa, Artificial Sequence
  Fragments of Bryodin 1
ALLLNRNNIAAIGED

SEQ ID NO: 179 - protein, 15 aa, Artificial Sequence
  Fragments of Bryodin 1
LNRNNIAAIGEDISM

SEQ ID NO: 180 - protein, 15 aa, Artificial Sequence
  Fragments of Bryodin 1
NNIAAIGEDISMTLI

SEQ ID NO: 181 - protein, 15 aa, Artificial Sequence
  Fragments of Bryodin 1
AAIGEDISMTLIGFE

SEQ ID NO: 182 - protein, 15 aa, Artificial Sequence
  Fragments of Bryodin 1
GEDISMTLIGFEHGL

SEQ ID NO: 183 - protein, 15 aa, Artificial Sequence
  Fragments of Bryodin 1
ISMTLIGFEHGLYGI